(19) United States
(12) Patent Application Publication
(10) Pub. No.: US 2003/0171347 A1
(43) Pub. Date: Sep. 11, 2003

(54) Sigma receptor antagonists having anti-cocaine properties and uses thereof

(76) Inventor: Rae R. Matsumoto, Edmond, OK (US)

Correspondence Address: DUNLAP, CODDING & ROGERS P.C., PO BOX 16370, OKLAHOMA CITY, OK, 73114, US

(21) Appl. No.: 10/178,859

(22) Filed: Jun. 21, 2002

Related U.S. Application Data
(63) Continuation of application No. 09/715,911, filed on Nov. 17, 2000, now abandoned.
Application No. 09/715,911 is a continuation of application No. 09/316,877, filed on May 21, 1999, now abandoned.
(60) Provisional application No. 60/086,550, filed on May 21, 1998.

(51) Int. Cl. A61K 31/55; A61K 31/33; A61K 31/397; A61K 31/445; A61K 31/40; A61K 31/137

(52) U.S. Cl. 514/183; 514/210.01; 514/217.12; 514/317; 514/408; 514/649

(57) ABSTRACT

The present invention relates to novel sigma receptor antagonist compounds that have anti-cocaine properties. These sigma receptor antagonists are useful in the treatment of cocaine overdose and addiction as well as movement disorders. The sigma receptor antagonists of the present invention may also be used in the treatment of neurological, psychiatric, gastrointestinal, cardiovascular, endocrine and immune system disorders as well as for imaging procedures. The present invention also relates to novel pharmaceutical compounds incorporating sigma receptor antagonists which can be used to treat overdose and addiction resulting from the use of cocaine and/or other drugs of abuse.

### CROSS-REFERENCE TO RELATED APPLICATIONS

This application is a continuation of U.S. patent application Ser. No. 09/715,911, filed Nov. 17,2000, entitled “SIGMA RECEPTOR ANTAGONISTS HAVING ANTI-COCAINE PROPERTIES AND USES THEREOF;” which is a continuation of U.S. patent application Ser. No. 09/316,877, filed May 21, 1999, entitled “SIGMA RECEPTOR ANTAGONISTS HAVING ANTI-COCAINE PROPERTIES AND USES THEREOF,” now abandoned; and claims priority under 35 U.S.C. §119(e) of U.S. Provisional Patent Application Serial No. 60/086,550, filed May 21, 1998, entitled “NOVEL SIGMA RECEPTOR LIGANDS FOR THE TREATMENT OF COCAINE ABUSE.”

### BACKGROUND OF THE INVENTION

1. Field of the Invention

The present invention relates to novel sigma receptor antagonist compounds that have anti-cocaine properties. These sigma receptor antagonists are useful in the treatment of cocaine overdose and addiction as well as movement disorders. The sigma receptor antagonists of the present invention may also be used in the treatment of neurological, psychiatric, gastrointestinal, cardiovascular, endocrine and immune system disorders as well as for imaging procedures. The present invention also relates to novel pharmaceutical compounds incorporating sigma receptor antagonists which can be used to treat overdose and addiction resulting from the use of cocaine and/or other drugs of abuse.

2. Brief Description of the Related Art

Cocaine has been reported to be the third most commonly abused drug, after alcohol and marijuana. It is further responsible for more serious intoxications and deaths than any other illicit compound. Currently, no effective treatments exist for cocaine overdose and addiction. Cocaine interacts with sigma receptors and this interaction provides the target for pharmacological intervention. Drugs that interfere with cocaine's access to sigma receptors mitigate the actions of cocaine. The sigma receptor compounds disclosed and claimed herein have anti-cocaine action: they prevent the behavioral toxic and psychomotor stimulant effects of cocaine.

Cocaine abuse and dependence have risen to epidemic proportions in recent years, and it remains a major public health problem. Cocaine has been reported to be the third most commonly abused drug, after alcohol and marijuana. In addition, cocaine overdose is responsible for more serious intoxications and deaths than any other illicit drug. Despite tremendous efforts in recent years to identify new treatment strategies to break the addictive process, existing treatments for cocaine addiction are very limited and there are no pharmacotherapies to address the problem of toxicity. In spite of the war on drugs, the prevalence of cocaine use remains high in the U.S. and as a consequence, there is an urgent need to develop effective pharmacotherapies to aid in breaking the cycle of abuse.

Past attempts to attenuate the toxic and addictive effects of cocaine have been met with limited success. Even in those instances where previously tested compounds have attenuated some of the actions of cocaine, major problems have persisted: ineffectiveness against the lethal effects of cocaine, ineffectiveness when administered after cocaine ingestion, unacceptable side effects, and/or a narrow therapeutic margin.

Cocaine is generally thought of as a dopamine uptake inhibitor. However, it can also inhibit the reuptake of serotonin and norepinephrine and bind to a number of neurotransmitter receptors. Of the myriad of sites with which cocaine interacts, the ones that are thought to be most relevant in terms of its psychological and physiological properties are the monoamine transporters, muscarinic receptors, and sigma receptors. It is at these sites that the affinity of cocaine corresponds to concentrations that are achievable in vivo.

Sigma receptor antagonists attenuate the behavioral toxic effects of cocaine, even when administered after an overdose and may also have prophylactic effects against the psychomotor stimulant effects of cocaine. These novel sigma receptor antagonists (“sigma ligands”) also have few side effects, wide therapeutic margins, and remain effective even when administered after a cocaine overdose.

Sigma receptors were first proposed in 1976 based on the actions of SKF 10,047 and related benzomorphans. The name “sigma” is in fact derived from the first letter “S” in SKF 10,047 which was thought to be the prototypic ligand for these binding sites. Unfortunately, SKF 10,047 is now recognized as a very non-selective ligand and some of the binding sites with which it interacts are shown in FIG. 1. In the very earliest experiments, investigators used racemic SKF 10,047, which is a mixture of the (+) and (−) forms of the drug. Later, when the isomers were tested, it was learned that the (−)-isomer predominantly interacts with what is today recognized as the kappa type of opiate receptor. The (+)-isomer, on the other hand, interacts with at least two sites: 1) PCP sites on the NMDA receptor, and it is due to this interaction that the term sigma-PCP was prevalent in the early 1980s; and 2) another site which today retains the designation of sigma.

Several lines of evidence support the physiological relevance of sigma receptors. First, there is evidence for endogenous ligands for these receptors. Several laboratories have independently isolated and reported brain extracts that are capable of binding to sigma receptors. In addition, endogenous factors that interact with sigma receptors can be released from specific brain pathways under more physiological conditions. In these latter studies, fresh slices of hippocampal brain sections were first incubated in physiological buffers with a radioligand to occupy sigma receptors. When the brain sections were depolarized using potassium chloride or veratridine, the radioligand that was bound to sigma receptors was displaced in a time- and calcium-dependent manner. Electrical stimulation of the perforant path and/or mossy fibers of the hippocampus produced similar effects, suggesting that depolarization of specific brain circuits caused the release of an endogenous substance that could then compete with the bound radioligand for sigma receptors. Together, the data indicate the existence of chemicals in the brain that bind to sigma receptors, and that can be liberated in specific brain circuits under conditions known to cause neurotransmitter release.

Second, there is evidence that sigma receptors are associated with the modulation or production of a number of second messengers including cGMP, inositol phosphates, G-proteins, and calcium. This coupling of sigma receptors with recognized effector systems is an important feature that would be expected of physiologically functioning receptors.

Third, the functional effects of sigma ligands, including the motor effects that are the focus of the present review, are correlated with their sigma binding affinities in a number of systems. Using such a correlational validation, sigma receptors have been implicated in motor effects, inhibition of contractions in the guinea pig ileum/myenteric plexus, inhibition of agonist-stimulated phosphoinositide turnover, and elicitation of an inward current in NCB-20 cells. Other actions for which there is strong evidence for a sigma receptor involvement although correlations have not yet been demonstrated include: learning and memory processes, and the modulation of NMDA and dopamine systems.

Fourth, a number of laboratories have now sequenced and cloned sigma receptor proteins. Recent studies using antisense oligodeoxynucleotides for these receptor sequences further confirm that knock down of the receptor protein can attenuate purported sigma-mediated behaviors. Similar to other receptors, biochemical studies demonstrate that there are multiple subtypes of sigma receptors, the most well characterized being the σ₁ and σ₂ sites. The major characteristics of the two sigma receptor subtypes are summarized in Table 1. Other less characterized subtypes also exist and any new subtypes that may be characterized in the future also fall within the broad scope and teachings of the present invention.

Although sigma receptors were initially thought to be a type of opiate receptor, these proteins can be distinguished from classical opiate (μ, 67 , κ) receptors because morphine, the opiate antagonist naloxone, and the endogenous opioid dynorphin are unable to bind to them. The ability of phencyclidine (PCP) to interact with sigma receptors also caused some initial confusion because PCP can also act within the ionophore of the NMDA receptor. Sigma receptors are nevertheless clearly distinct from the non-competitive sites on the NMDA receptor because NMDA receptor channel blockers such as MK-801 are unable to interact with them. Sigma receptors can also be distinguished from dopamine receptors because dopamine itself is inactive. Therefore, although sigma receptors interact with many compounds that bind dopamine receptors, opiate receptors, and non-competitive sites on NMDA receptors, when the overall drug selectivity pattern is considered, sigma receptors can be distinguished from these previously characterized binding sites.

Sigma receptors are particularly attractive targets for drug development efforts because they are localized in key organ systems (i.e. brain, heart, lung) that are involved in multiple stages and aspects of cocaine's actions. Cocaine is known to interact with several binding sites (e.g. monoamine transporters, sigma receptors, and muscarinic receptors) at concentrations that are achievable in vivo. Subsequent to the binding of cocaine to these proteins, a complex, cascade of events occurs. Sigma receptors are involved in several critical stages of this cascade.

Following the administration of reinforcing doses of cocaine, mesocorticolimbic dopamine systems are activated. The activation of these systems is thought to be responsible for the reinforcing properties of cocaine and other psychomotor stimulants, and to contribute to the vulnerability to drug addiction. Although the activation of mesocorticolimbic dopamine systems is generally thought to result from cocaine's blockade of the dopamine transporter, it may also be triggered as a consequence of cocaine's actions through sigma receptors. The interaction between sigma and dopamine systems is well documented, including the ability of sigma receptors agonists to cause dopamine synthesis and release.

In the case of cocaine overdose, an important downstream event is the overactivation of N-methyl-D-aspartate (NMDA) receptors. As a consequence of NMDA receptor dysfunction, the regulation of neural, cardiovascular, and respiratory functions become compromised. Although the relationship between sigma and NMDA systems are not as well defined as the interaction with dopamine systems, the ability of sigma ligands to modulate NMDA-mediated responses is also well documented. Cocaine also has local anesthetic effects, which may be influenced by sigma receptors. Theres is a correlation between the ability of cocaine congeners to bind to σ receptors and their binding to sites on Na+channels that are important in local anesthetic effects.

The presence of sigma receptors in multiple, critical stages in the physiological cascade following exposure to cocaine provides a logical explanation for the dramatic therapeutic effects of the novel sigma ligands described and claimed herein, when compared to previously tested monoamine transporter and muscarinic antagonists which appear to have their greatest impact early in the cascade. Such a multi-stage and systems approach with our sigma ligands is reminiscent of the actions of opiate antagonists and partial agonists for the treatment of opiate addiction and overdose, where opiate receptors are involved not only early in the cascade, but also at critical downstream events (e.g. opiate-dopamine interactions, respiratory depression).

The sigma ligands described and claimed herein possess moderate to high affinity for their target sites (sigma receptors), and low to negligible affinities for other sites with which non-selective sigma ligands have a tendency to interact (e.g. dopaminergic, opiatergic, PCP, muscarinic, adrenergic, serotonergic). These sigma ligands also possess functional antagonistic activity. The first series of sigma ligand compounds can be generally divided into three groups, relative to the parent compound BD1008: (1) N-alkyl substituted compounds and those with miscellaneous alterations; (2) conformationally restricted compounds; and (3) mono-aryl substituted compounds. The second series of sigma ligand compounds generally fall into a category which has significantly more substitutions and modifications of the BD1008 parent compound.

Thus it is an object of the present invention to provide novel sigma receptor antagonists for use in the treatment of cocaine addiction as well as other physical and mental ailments. These and other objects of the present invention will become apparent in light of the present specification, claims, and drawings.

### SUMMARY OF THE INVENTION

The present invention relates to novel sigma receptor antagonist compounds that have anti-cocaine properties. These sigma receptor antagonists are useful in the treatment of cocaine overdose and addiction as well as movement disorders. The sigma receptor antagonists of the present invention may also be used in the treatment of neurological, psychiatric, gastrointestinal, cardiovascular, endocrine and immune system disorders as well as for imaging procedures. The present invention also relates to novel pharmaceutical compounds incorporating sigma receptor antagonists which can be used to treat overdose and addiction resulting from the use of cocaine and/or other drugs of abuse.

### BRIEF DESCRIPTION OF THE SEVERAL VIEWS OF THE DRAWINGS

FIG. 1 is a diagrammatical representation of the hypothesized sequence of events after exposure.

FIG. 2 is a diagrammatical representation of the dose response for the behavioral toxic effects of cocaine.

FIG. 3 is a diagrammatical representation of the dose response for the locomotor stimulatory effects of cocaine.

FIG. 4 is a diagrammatical representation of the N-alkyl substituted compounds which attenuated cocaine-induced convulsions.

FIG. 5 is a diagrammatical representation of the pyrrolidinyl ring altered compounds which attenuate cocaine-induced convulsions.

FIG. 6 is a diagrammatical representation of the conformationally restricted compounds which attenuate cocaine-induced convulsions.

FIG. 7 is a diagrammatical representation of the aryl monosubstituted compounds which attenuate cocaine-induced convulsions.

FIG. 8 is a diagrammatical representation of the historic “sigma” compounds showing that these compounds vary in their ability to attenuate cocaine-induced convulsions.

FIG. 9 is a diagrammatical representation showing that the sigma receptor agonists worsen cocaine-induced convulsions.

FIG. 10 is a diagrammatical representation of the N-alkyl substituted compounds which attenuate cocaine-induced lethality.

FIG. 11 is a diagrammatical representation of the pyrrolidinyl ring altered compounds which attenuate cocaine induced lethality.

FIG. 12 is a diagrammatical representation of the conformationally-restricted compounds which attenuate cocaine-induced lethality.

FIG. 13 is a diagrammatical representation of the aryl monosubstituted compounds which attenuate cocaine-induced lethality.

FIG. 14 is a diagrammatical representation showing that the historic “sigma” compounds vary in their ability to attenuate cocaine-induced lethality.

FIG. 15 is a diagrammatical representation showing that sigma receptor agonists fail to attenuate cocaine-induced lethality.

FIG. 16 is a diagrammatical representation showing that post-treatment with N-alkyl substituted compounds did not attenuate cocaine-induced lethality.

FIG. 17 is a diagrammatical representation showing that post-treatment with conformationally restricted compounds varied in their ability to attenuate cocaine-induced lethality.

FIG. 18 is a diagrammatical representation showing that post-treatment with a pyrrolidinyl ring altered compound did not attenuate cocaine-induced lethality.

FIG. 19 is a diagrammatical representation showing that post-treatment with aryl monosubstituted compounds attenuated cocaine-induced lethality.

FIG. 20 is a diagrammatical representation showing the effects of N-alkyl substituted ligands on baseline locomotor activity.

FIG. 21 is a diagrammatical representation showing the effects of pyrrolidinyl ring altered ligands on baseline locomotor activity.

FIG. 22 is a diagrammatical representation showing the effects of conformationally restricted ligands on baseline locomotor activity.

FIG. 23 is a diagrammatical representation showing the effects of aryl monosubstituted ligands on baseline locomotor activity.

FIG. 24 is a diagrammatical representation showing that N-alkyl substituted ligands attenuate cocaine-induced locomotor activity.

FIG. 25 is a diagrammatical representation showing that pyrrolidinyl ring altered ligands attenuate cocaine-induced locomotor activity.

FIG. 26 is a diagrammatical representation showing that conformationally restricted ligands attenuate cocaine-induced locomotor activity.

FIG. 27 is a diagrammatical representation showing that aryl monosubstituted ligands attenuate cocaine-induced locomotor activity.

FIG. 28 is a diagrammatical representation showing that the sigma ligands described and claimed herein shift the dose curve for the locomotor stimulatory effects of cocaine to the right.

FIG. 29 is a diagrammatical representation showing the NYU antisense oligodeoxynucleotide against σ₁ receptors attenuates cocaine-induced convulsions.

FIG. 30 is a diagrammatical representation showing that the McGill antisense oligodeoxynucleotide against σ₁ receptors attenuates cocaine-induced convulsions.

FIG. 31 is a diagrammatical representation showing that treatment with antisense oligodeoxynucleotide has no effect on basal locomotor activity.

FIG. 32 is a diagrammatical representation showing that the NYU antisense oligodeoxynucleotide against σ₁ receptors attenuates the locomotor stimulatory effects of cocaine.

FIG. 33 is a diagrammatical representation showing that the McGill antisense oligodeoxynucleotide against σ₁ receptors attenuates the locomotor stimulatory effects of cocaine.

FIG. 34 is a diagrammatical representation showing that the relationship between σ₁ binding and attenuation of cocaine-induced convulsions.

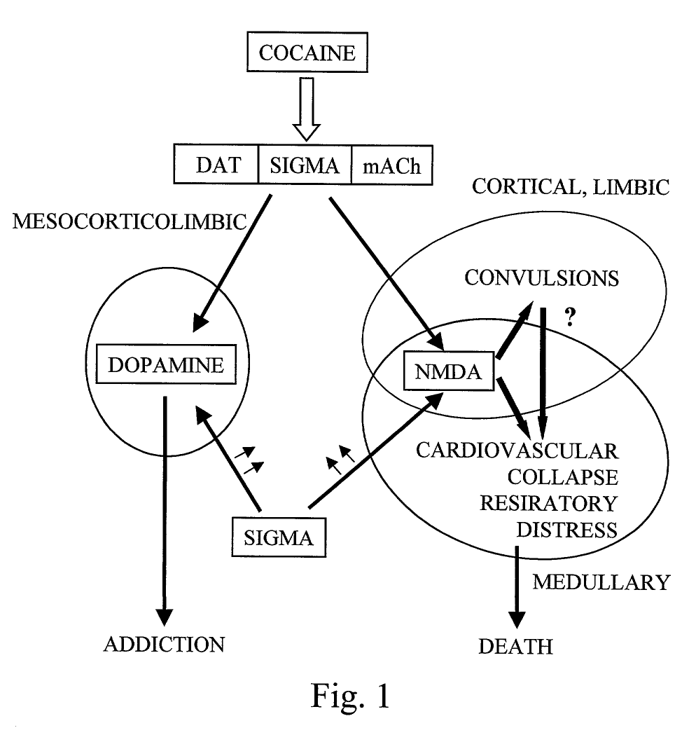

### DETAILED DESCRIPTION OF THE INVENTION

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The present invention relates to novel sigma receptor antagonist compounds that have anti-cocaine properties. These sigma receptor antagonists are useful in the treatment of cocaine overdose and addiction as well as movement disorders. The sigma receptor antagonists of the present invention may also be used in the treatment of neurological, psychiatric, gastrointestinal, cardiovascular, endocrine and immune system disorders as well as for imaging procedures. The present invention also relates to novel pharmaceutical compounds incorporating sigma receptor antagonists which can be used to treat overdose and addiction resulting from the use of cocaine and/or other drugs of abuse.

In spite of extensive efforts in the field of addiction and overdose treatment, the lack of pharmacotherapies to manage cocaine overdose situations persists as a major void in the ongoing efforts to overcome the lasting effects of cocaine. A strategy for developing an effective anti-cocaine agent is to block cocaine's access to the receptors at which it acts. The ability of cocaine to interact with sigma receptors at concentrations that are achievable in vivo has been known for the past ten years. Earlier efforts to target these sites, however, were met with mixed results, and little has since been done to follow up on the previous efforts. One of the major problems with targeting sigma receptors was the lack of selective and potent pharmacological and molecular biological tools with which to probe this system. Our sigma receptor ligands produce anti-cocaine action by. antagonizing sigma receptors.

The first series of sigma receptor compounds described and claimed herein are analogs of BD1008 (N-[2-(3,4-dicholorphenyl)ethyl]-N-methyl-2(1-pyrrolidinyl)ethylamine) with several modifications: N-alkyl substitutions, pyrrolidinyl ring alterations, conformational restriction, aryl, and other monosubstitutions. The second series of sigma receptor compounds have significant more substitutions and modifications to the parent BD1008 compound. Although most compounds which have historically been labeled “sigma ligands” tend to be non-specific, the sigma ligands described and claimed herein are relatively selective for sigma receptors. The parent compound BD1008, as well as the analogs, have high affinities for sigma receptors, but low to negligible affinities for nine other tested sites with which non-specific “sigma ligands” interact (dopamine, opiate, PCP, muscarinic, α₁, α₂, μ, 5-HT₁, 5-HT₂). Table 1 provides the structures and R group substitutions for the sigma ligands described and claimed herein as well as other relevant compounds and controls.

TABLE 1
First series of anti-cocaine compounds

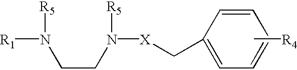

| | | | | | | |
|---|---|---|---|---|---|---|
| R₁ | R₂ | R₃ | R₄ | X | σ₁ | σ₂ |

Parent:

| | | | | | | |
|---|---|---|---|---|---|---|
| BD1008 | —(CH₂)₅— | Me | 3,4-dichloro | CH₂ | 2 ± 1 | 8 ± 2 |

N-alkyl substituted compounds:

| | | | | | | |
|---|---|---|---|---|---|---|
| BD1060 | —(CH₂)₅— | H | 3,4-dichloro | CH₂ | 3 ± 0.1 | 156 ± 45 |
| BD1067 | —(CH₂)₅— | Et | 3,4-dichloro | CH₂ | 2 ± 0.5 | 39 ± 1 |
| BD1069* | —(CH₂)₅— | n-Pr | 3,4-dichloro | CH₂ | 1 ± 0.2 | 545 ± 139 |
| BD1052* | —(CH₂)₅— | allyl | 3,4-dichloro | CH₂ | 2 ± 0.5 | 60 ± 3 |

Pyrrolidino ring altered compounds:

| | | | | | | | |
|---|---|---|---|---|---|---|---|
| BD1047 | Me | Me | Me | 3,4-dichloro | CH₂ | 0.9 ± 0.14 | 7 ± 0.6 |
| LR172 | —(CH₂)₆— | | Me | 3,4-dichloro | CH₂ | 0.4 ± 0.09 | 2 ± 0.3 |
| BD1063 | Me | —(CH₂)₂— | | 3,4-dichloro | CH₂ | 9 ± 1 | 449 ± 11 |

Aryl monosubstituted compounds:

| | | | | | | |
|---|---|---|---|---|---|---|
| YZ-005* | —(CH₂)₅— | Me | m-methoxy | C═O | 3101 ± 20 | 1114 ± 55 |
| YZ-007* | —(CH₂)₅— | Me | p-methoxy | C═O | 2223 ± 61 | 1215 ± 95 |
| YZ-008* | —(CH₂)₅— | Me | o-methoxy | C═O | 5457 ± 686 | 3170 ± 144 |
| YZ-011 | —(CH₂)₅— | Me | m-methoxy | CH₂ | 24 ± 2 | 209 ± 22 |
| YZ-016 | —(CH₂)₅— | Me | p-methoxy | CH₂ | 30 ± 1 | 256 ± 2 |
| YZ-018 | —(CH₂)₅— | Me | o-methoxy | CH₂ | 16 ± 2 | 144 ± 1 |
| YZ-027 | —(CH₂)₅— | Me | m-nitro | CH₂ | 6 ± 2 | 95 ± 0.7 |
| YZ-028 | —(CH₂)₅— | Me | o-nitro | CH₂ | 8 ± 0.9 | 311 ± 4 |
| YZ-029 | —(CH₂)₅— | Me | p-nitro | CH₂ | 4 ± 0.9 | 63 ± 0.1 |
| YZ-030 | —(CH₂)₅— | Me | p-amine | CH₂ | 276 ± 13 | 973 ± 77 |
| YZ-032 | —(CH₂)₅— | Me | o-amine | CH₂ | 291 ± 38 | 640 ± 25 |
| YZ-033 | —(CH₂)₅— | Me | m-amine | CH₂ | 579 ± 35 | 2235 ± 260 |

Conformationally restricted compounds:

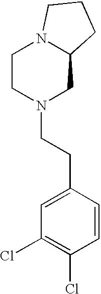

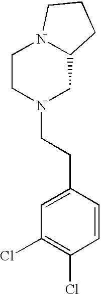

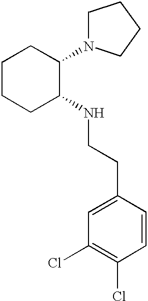

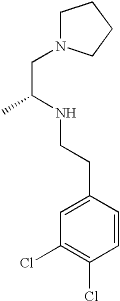

| | σ₁ | σ₂ |
|---|---|---|
| BD1018 | 5 ± 0.7 | 49 ± 4 |
| BD1031* | 1 ± 0.2 | 80 ± 9 |
| BD1063 | 9 ± 1 | 449 ± 11 |
| LR132 | 2 ± 0.1 | 701 ± 375 |
| LR176 | 2 ± 0.01 | 44 ± 2 |

Second Series of Anti-Cocaine Compounds

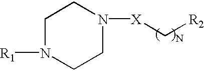

| | R1 | R2 | X | σ1 | σ2 |
|---|---|---|---|---|---|
| YZ-067-2 | Ph(CH₂)₃ | p-methoxyphenyl | CH₂ | 1 ± 0.3 | 29 ± 2 |
| YZ-184-2 | Ph(CH₂)₃ | o-methoxyphenyl | CH₂ | 4 ± 0.6 | 8 ± 0.2 |
| YZ-069-2 | Ph(CH₂)₃ | 3,4-dichlorophenyl | CH₂ | 2 ± 0.2 | 39 ± 2 |
| YZ-048-2 | Ph(CH₂)₃ | 0-methoxyphenyl | C═O | 56 ± 8 | 294 ± 10 |
| YZ-051-2 | Ph(CH₂)₃ | p-methoxyphenyl | C═O | 13 ± 2 | 464 ± 34 |
| YZ-085-2 | m-methoxy- | m-methoxyphenyl | C═O | n.d. | 12 ± 2 |
| | phenylethyl | | | | |
| YZ-136-2 | Ph(CH₂)₃ | NH₂ | CH₂ | 3 ± 0.09 | 188 ± 13 |
| YZ-155-2 | Ph(CH₂)₃ | p-nitrophenyl | C═O | 14 ± 1 | 167 ± 4 |
| YZ-165-2-2 | Ph(CH₂)₃ | o-aminophenyl | CH₂ | 5 ± 0.1 | 18 ± 0.2 |
| YZ-166-2-2 | Ph(CH₂)₃ | m-aminophenyl | CH₂ | 2 ± 0.3 | 37 ± 1 |
| (N =1 for all) | | | | | |
| Traditional: | | | | | |
| Haloperidol* | | | | 4 ± 0.6 | 12 ± 2 |
| Reduced | | | | 11 ± 0.3 | 32 ± 4 |
| haloperidol* | | | | | |
| DTG* | | | | 28 ± 4 | 13 ± 2 |
| (+)-Penta- | | | | 3 ± 0.3 | 1542 ± 313 |
| zocine* | | | | | |
| Rimcazole* | | | | 2380 ± 812 | 1162 ± 160 |
| BMY14802* | | | | 66 ± 11 | 51 ± 8 |
| Naloxone* | | | | >10,000 | >10,000 |

(*items were the controls).

Although receptor binding studies indicate the extent to which ligands interact with sites of interest, they provide no information about the nature of the actions of the compounds once they bind to the receptor. Thus sigma ligands must be assigned agonist versus antagonist designations based on pharmacological definitions. Therefore, compounds that bind to the receptor and produce actions on their own are designated as agonists. Compounds that bind to the receptor, produce no actions on their own, but have the ability to attenuate the actions of an “agonist” are considered antagonists.

An important consideration when applying such designations is the use of an appropriate functional system in which to screen for agonist versus antagonist effects. Prior to use in the cocaine experiments, the compounds were characterized in an animal model of acute dystonic reaction in which abnormal head angles resulted after microinjection of sigma ligands into the rat red nucleus. The reaction was chosen for this initial testing because the red nucleus contains a high concentration of sigma receptors as compared to the paucity of other receptors with which non-selective sigma ligands interact. This behavior is known to be mediated through sigma receptors because: (1) the ED₅₀s of sigma ligands for producing this behavior are significantly correlated with their sigma binding affinities and (2) non-sigma compounds, such as selective ligands for dopamine, opiate, PCP, adrenergic, muscarinic, and serotonergic receptors are unable to produce this behavior. Utilizing this functional system, the sigma ligands that produce dystonic head postures were designated as agonists. Other compounds were designated as antagonists because they have high affinity for sigma receptors, produce no effects on their own when microinjected into the rat red nucleus, but attenuate the dystonic head postures produced by other sigma agonists.

Based on these classifications and studies, the traditional sigma ligands DTG and (+)-pentazocine act as agonists. The novel sigma ligands BD1052 and BD1031 also possess agonist actions, while BD1069 possesses variable, partial agonist actions. In contrast, BD1047 and BD1063 act as functional antagonists. They produce no effects on their own, but have the ability to attenuate the dystonic postures elicited by sigma receptor agonists such as DTG. The sigma ligands BD1067 and YZ-016 also appear to possess antagonistic actions, because they produce the same pattern of results in these rat dystonia experiments as BD1047 and BD1063.

The sigma ligands described and claimed herein have high affinities for sigma receptors and negligible affinities for other receptors with which the traditional “sigma ligands” interact, which are shown in Table 1. The affinities of the ligands for the two most well characterized sigma receptor subtypes were determined using methods previously published in detail by Bowen W D, de Costa B R, Hellewell S B, Walker J M, Rice K C (1993) [³H](+)-Pentazocine: a potent and highly selective benzomorphan-based probe for sigma-1 receptors, Mol Neuropharmacol 3:117-126, and Matsumoto R R, Bowen W D, Tom M A, Vo V N, Truong D D, de Costa B R (1995) Characterization of two novel σ receptor ligands: antidystonic effects in rats suggest σ receptor antagonism, Eur J Pharmacol 280:301-310, the contents of which are here in wholly incorporated by reference. Briefly, the affinities were determined under conditions that most selectively label the putative sigma receptor subtypes: i.e. σ₁ receptors were labeled with [³H](+)-pentazocine in guinea pig brain; σ₂ sites were labeled in rat liver with [³ H]di-o-tolylguanidine (DTG) in the presence of a saturating concentration of dextrallorphan. Non-specific binding was determined with haloperidol. The values given in Table 1 represent the mean±SEM(K₁ in nM) of a minimum of two independent assays.

Since many historic “sigma ligands” have non-specific interactions with dopamine (D₂) receptors, opiate receptors, and the PCP site on NMDA receptors, the affinities of the sigma ligands described and claimed herein for these sites were determined. In addition, the affinities of the ligands for 5-HT₂ receptors were of interest because antagonism of these receptors can attenuate the convulsive effects of cocaine. The competition binding assays were performed in homogenates of rat brain using methods that have been previously published in detail by Matsumoto R R, Bowen W D, Tom M A, Vo V N, Truong D D, de Costa B R (1995) Characterization of two novel σ receptor ligands: antidystonic effects in rats suggest σ receptor antagonism, Eur J Pharmacol 280:301-310, the contents of which are herein wholly incorporated by reference. Dopamine receptors were labeled with [³H](−)sulpiride, opiate receptors were labeled with [³H]etorphine or [³H]bremazocine, PCP sites on NMDA receptors were labeled with [³H]1-[(2-thienyl)-cyclohexyl]piperidine (TCP), and 5-HT₂ receptors were labeled with [³H] ketanserin. The numbers listed in Table 2 represent IC₅₀ values in nM.

TABLE 2

| | Dopamine (D₂) | Opiate | NMDA (PCP) | 5-HT₂ |
|---|---|---|---|---|
| Parent: | | | | |
| BD1008 | 1112 ± 74 | >10,000 | >10,000 | >10,000 |

N-alkyl substitutions:

| | | | | |
|---|---|---|---|---|
| BD1060 | >10,000 | >10,000 | >10,000 | >10,000 |
| BD1067 | >10,000 | >10,000 | >10,000 | >10,000 |
| BD1069* | >10,000 | >10,000 | >10,000 | >10,000 |
| BD1052* | >10,000 | >10,000 | >10,000 | >10,000 |

Pyrrolidino ring alterations:

| | | | | |
|---|---|---|---|---|
| BD1047 | >10,000 | >10,000 | >10,000 | 4667 ± 2626 |
| LR172 | >10,000 | >10,000 | >10,000 | >10,000 |

Aryl monosubstitutions:

| | | | | |
|---|---|---|---|---|
| YZ-005* | >10,000 | >10,000 | >10,000 | >10,000 |
| YZ-007* | >10,000 | >10,000 | >10,000 | >10,000 |
| YZ-008* | >10,000 | >10,000 | >10,000 | >10,000 |
| YZ-011 | >10,000 | >10,000 | >10,000 | >10,000 |
| YZ-016 | >10,000 | >10,000 | >10,000 | >10,000 |
| YZ-018 | >10,000 | >10,000 | >10,000 | 4441 ± 541 |
| YZ-027 | >10,000 | >10,000 | >10,000 | >10,000 |
| YZ-028 | >10,000 | >10,000 | >10,000 | >10,000 |
| YZ-029 | >10,000 | >10,000 | >10,000 | >10,000 |
| YZ-030 | >10,000 | >10,000 | >10,000 | >10,000 |
| YZ-032 | >10,000 | >10,000 | >10,000 | >10,000 |
| YZ-033 | >10,000 | >10,000 | >10,000 | >10,000 |

Conformationally restricted:

| | | | | |
|---|---|---|---|---|
| BD1018 | >10,000 | >10,000 | >10,000 | 1246 ± 14 |
| BD1031* | >10,000 | >10,000 | >10,000 | 2670 ± 769 |
| BD1063 | >10,000 | >10,000 | >10,000 | 2552 ± 2417 |
| LR132 | >10,000 | >10,000 | >10,000 | >10,000 |
| LR176 | >10,000 | >10,000 | >10,000 | >10,000 |

(*items denote controls)

All of the compounds have a 100-fold or lower affinity for these non-sigma sites (dopamine, opiate, NMDA, 5-HT₂) as compared to sigma receptors.

In order to further evaluate the relative selectivities of the ligands described and claimed herein for sigma receptors, their affinities for α₁, α₂-, β-adrenergic, 5-HT₁, and muscarinic cholinergic receptors were also determined for a select group of compounds. The competition binding assays were performed in homogenates of rat brain using methods that have been previously published in detail in Matsumoto R R, Bowen W D, Tom M A, Vo V N, Truong D D, de Costa B R (1995) Characterization of two novel σ receptor ligands: antidystonic effects in rats suggest σ receptor antagonism, Eur J Pharmacol 280:301-310 the contents of which are wholly incorporated by reference herein. The α₁ -adrenoceptors were labeled with [³ H]prazosin, α₂-adrenoceptors were labeled with [³H]clonidine, β-adrenoceptors were labeled with [³H]dihydroalprenolol, 5-HT₁ receptors were labeled with [³H] 5-HT, and muscarinic cholinergic receptors were labeled with [³H] pirenzepine. The numbers listed in Table 3 below represent IC₅₀ values in nM.

TABLE 3

| | α₁ | α₂ | β | 5-HT₁ | mACh |
|---|---|---|---|---|---|
| Parent: | | | | | |
| BD1008 | >10,000 | >10,000 | 6783 ± 1861 | >10,000 | 5,000 |
| N-alkyl substitutions: | | | | | |
| BD1052* | >10,000 | >10,000 | >10,000 | >10,000 | n.d. |
| Pyrrolidino ring alterations: | | | | | |
| BD1047 | >10,000 | >10,000 | 145 ± 100 | >10,000 | 8000 ± 1000 |
| Conformationally restricted: | | | | | |
| BD1018 | >10,000 | 6979 ± 4332 | >10,000 | >10,000 | 2000 |
| BD1031* | >10,000 | >10,000 | 7258 ± 3537 | >10,000 | 1670 |
| BD1063 | >10,000 | >10,000 | >10,000 | >10,000 | 9460 ± 750 |

(*items denote controls)

In addition to the core group of compounds described thus far, a second series of novel ligands were tested which have more extensive modifications to the BD1008 structure. The affinities of these ligands for the two most well characterized sigma receptor subtypes were determined using methods previously referenced above. The affinities were determined under conditions that most selectively label the putative sigma receptor subtypes. The σ₁ receptors were labeled with [3H](+)-pentazocine in guinea pig brain; σ₂ sites were labeled in rat liver with [³H]di-o-tolylguanidine (DTG) in the presence of a saturating concentration of dextrallorphan. Non-specific binding was determined with haloperidol. The values listed in Table 4 are in Kᵢ in nM. The “n” can be any integer from 1 to 5.

TABLE 4

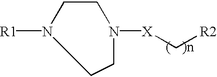

| | R₁ | R₂ | X | σ₁ | σ₂ |
|---|---|---|---|---|---|
| YZ-067-2 | Ph(CH₂)₃ | p-methoxyphenyl | CH₂ | 1 ± 0.3 | 29 ± 2 |
| YZ-184-2 | Ph(CH₂)₃ | o-methoxyphenyl | CH₂ | 4 ± 0.6 | 8 ± 0.2 |
| YZ-069-2 | Ph(CH₂)₃ | 3,4-dichlorophenyl | CH₂ | 2 ± 0.2 | 39 ± 2 |
| YZ-048-2 | Ph(CH₂)₃ | o-methoxyphenyl | C═O | 56 ± 8 | 294 ± 10 |
| YZ-051-2 | Ph(CH₂)₃ | p-methoxyphenyl | C═O | 13 ± 2 | 464 ± 34 |
| YZ-085-2 | m-methoxy- | m-methoxyphenyl | C═O | n.d. | 12 ± 2 |
| | phenylethyl | | | | |
| YZ-136-2 | Ph(CH₂)₃ | NH₂ | CH₂ | 3 ± 0.09 | 188 ± 13 |
| YZ-155-2 | Ph(CH₂)₃ | p-nitrophenyl | C═O | 14 ± 1 | 167 ± 4 |
| YZ-165-2-2 | Ph(CH₂)₃ | o-aminophenyl | CH₂ | 5 ± 0.1 | 18 ± 0.2 |
| YZ-166-2-2 | Ph(CH₂)₃” | o-aminophenyl | CH₂ | 2 ± 0.3 | 37 ± 1 |

(N = 1 in all the above compounds)

It is unlikely that the sigma ligands described and claimed herein interact with the dopamine transporter because BD1008, the parent compound, is inactive at these sites (>100,000 nM). However, some compounds that are generally thought of as ligands that target the dopamine transporter have significant affinities for sigma receptors. It is likely that some of these compounds act as antagonists at sigma receptors. The values listed below in Table 5 are in Kᵢ in nM.

TABLE 5

| | σ₁ | σ₂ |
|---|---|---|
| GBR 12909 | 132 ± 31 | 133 ± 37 |
| Benztropine | 403 ± 107 | 31 ± 4 |

In terms of mixed compounds with dopamine transporter activity and sigma binding affinity, and GBR analogs all have varying degrees of affinity ratios for sigma receptors versus dopamine transporters. The structures of these compounds are shown in Table 6.

TABLE 6

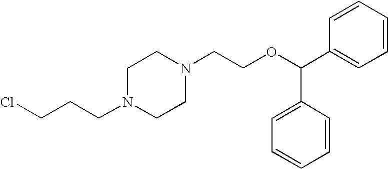

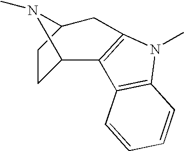

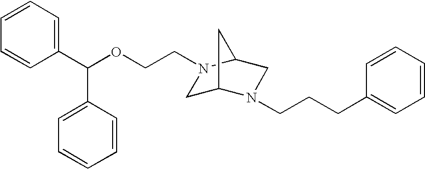

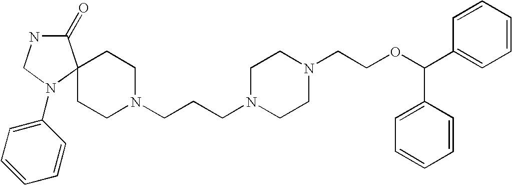

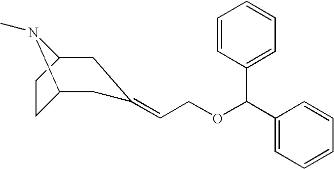

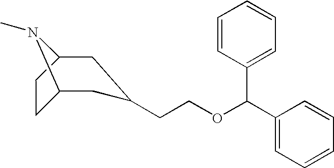

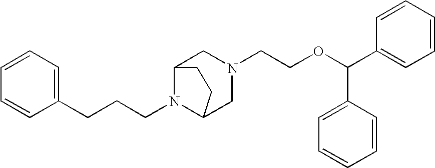

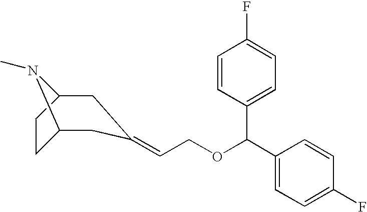

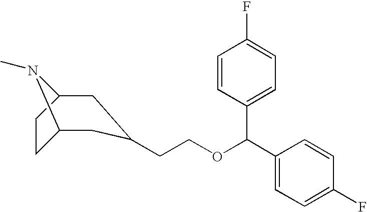

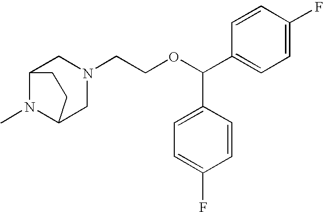

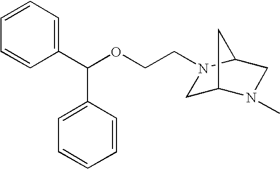

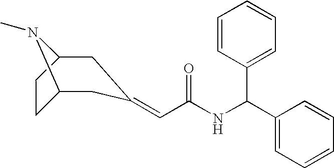

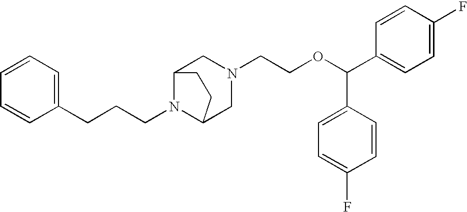

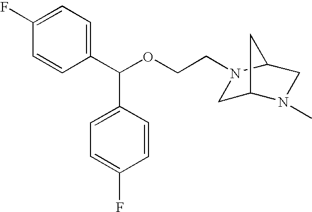

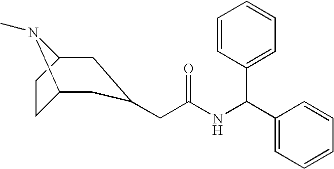

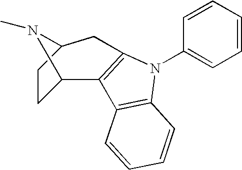

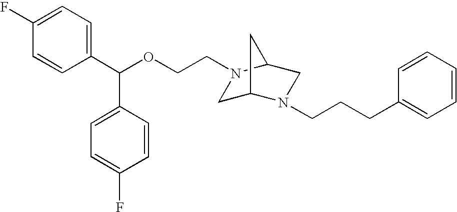

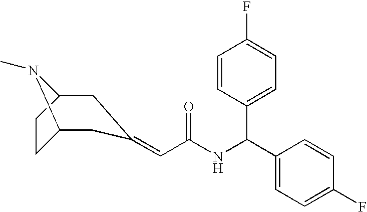

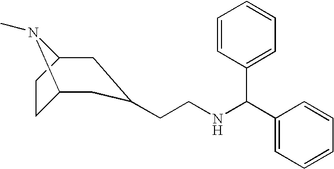

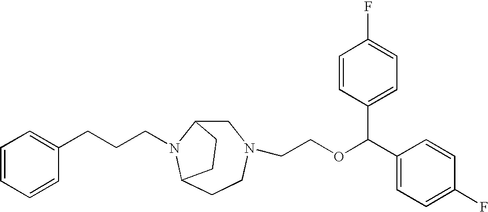

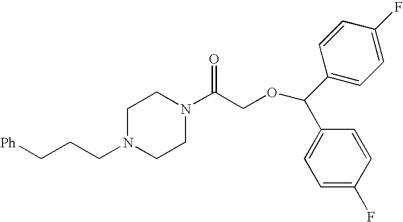

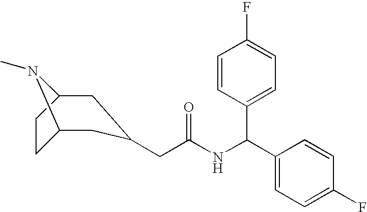

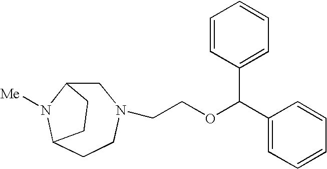

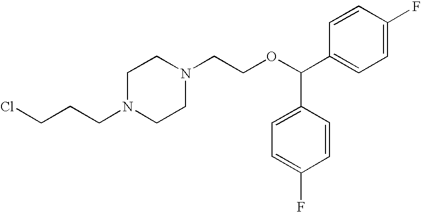

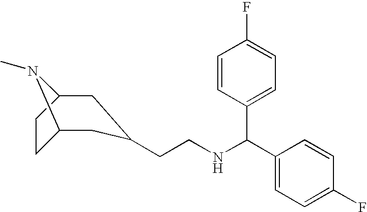

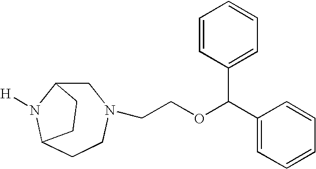

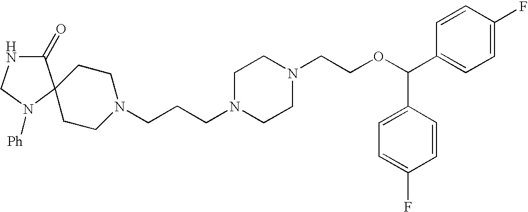

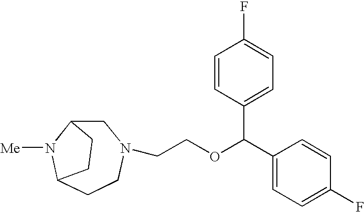

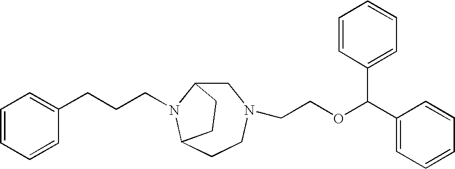

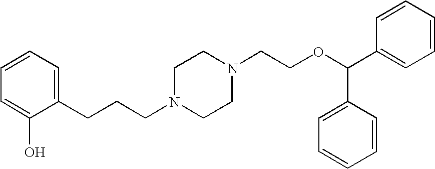

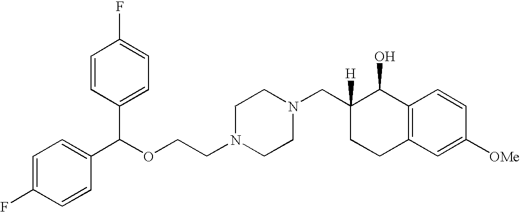

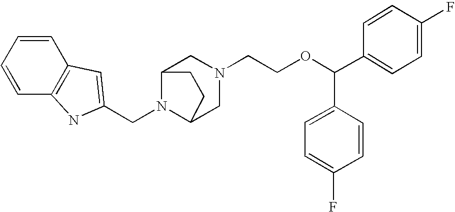

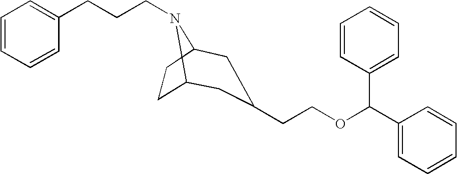

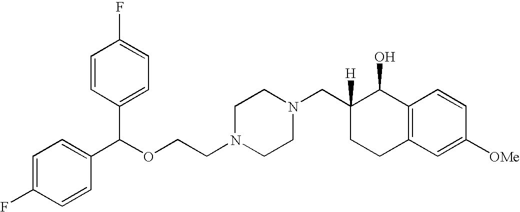

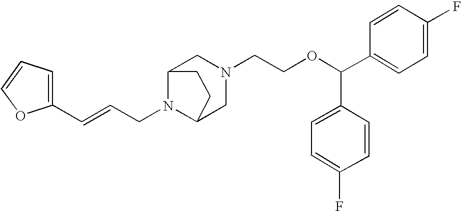

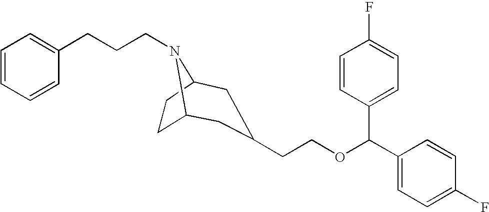

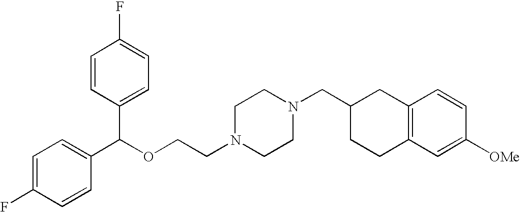

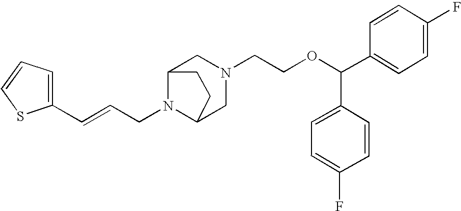

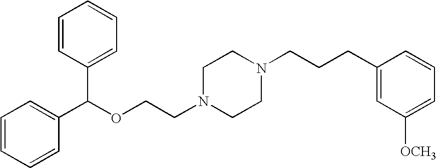

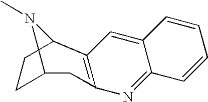

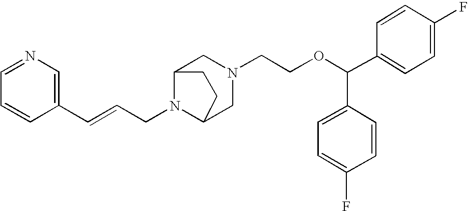

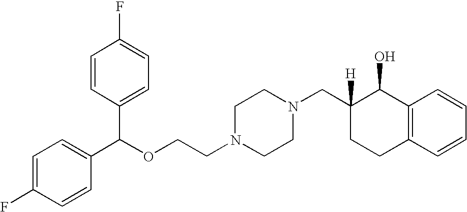

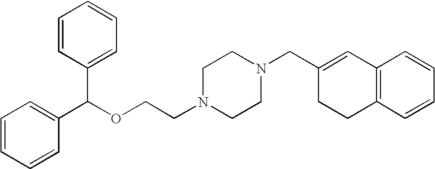

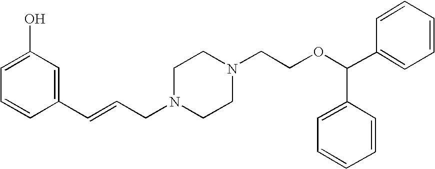

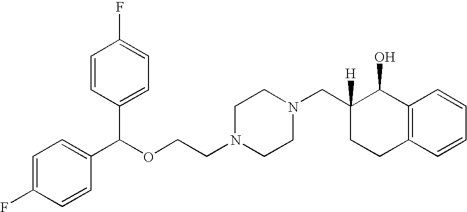

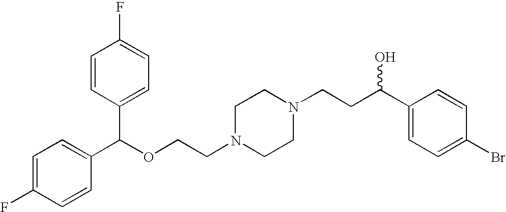

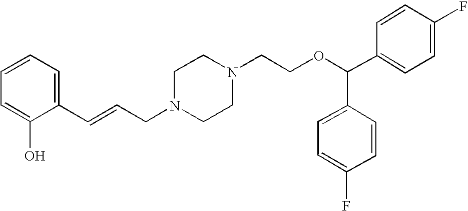

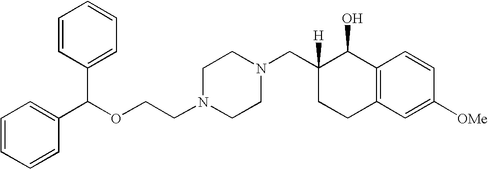

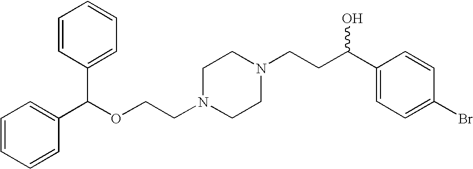

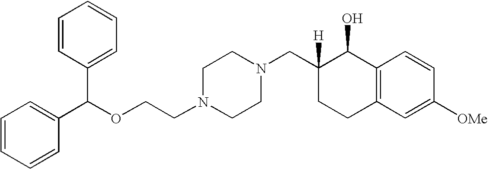

In order to test the dose response for the behavioral toxic effect of cocaine, male, Swiss Webster mice were injected (i.p.) with a dose of cocaine (0-150 mg/kg) and observed for the next 30 min for the onset of convulsions or death. Convulsions were operationally defined as a loss of righting reflexes for at least 5 sec combined with the presence of clonic limb movements. The number of mice convulsing or dying out of the total number of mice tested for each treatment group was noted.

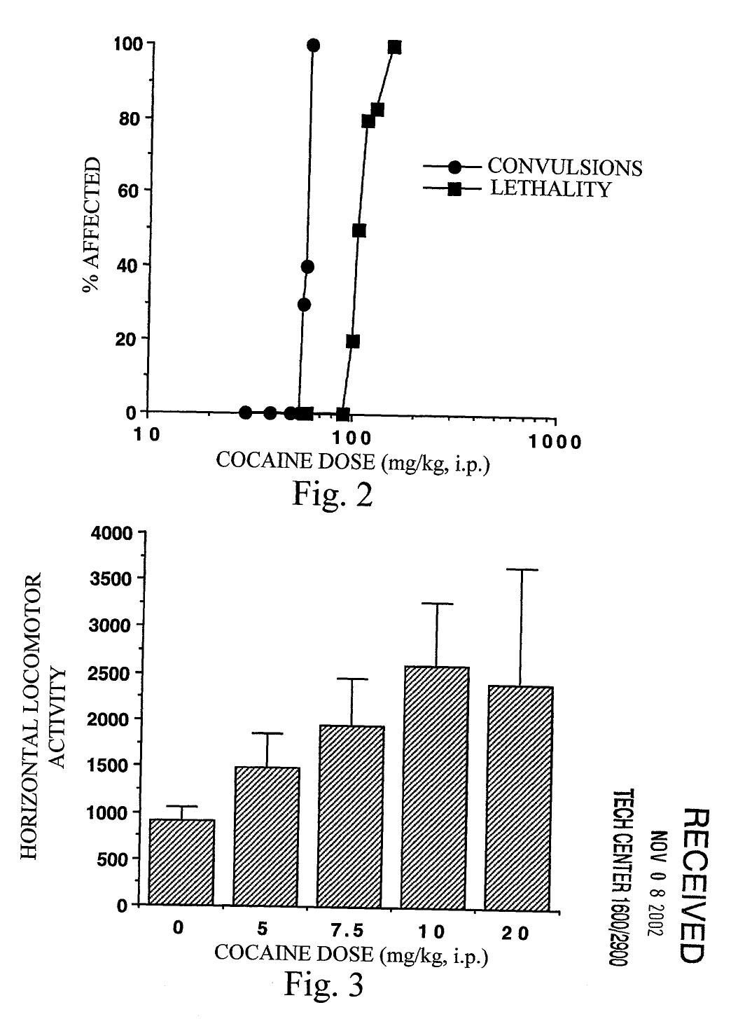

There was a dose-dependent increase in the proportion of mice exhibiting convulsions or death after receiving cocaine. The calculated ED₅₀ value for cocaine-induced convulsions was 59 mg/kg, i.p. The 60 mg/kg, i.p. dose of cocaine that was used as the challenge dose in the antagonism portions of the study therefore corresponded to the calculated ED₉₇ dose. The LD₅₀ value for cocaine-induced lethality was 108 mg/kg, i.p. The 125 mg/kg, i.p. challenge dose of cocaine that was used in the antagonism portion of the study thus represented the calculated LD₉₇ dose. As depicted in FIG. 2, the dose curve for both cocaine-induced convulsions and death was extremely steep.

Pre-treatment of mice with vehicle (saline) prior to administration of a convulsive dose of cocaine had no effect; 100% of the mice exhibited cocaine-induced convulsions. In contrast, pre-treatment of the mice with BD1008 or one of its analogs reliably attenuated cocaine-induced convulsions at effective doses. BD1008 and its N-alkyl substituted analogs (BD1060, BD1067) significantly reduced the number of mice exhibiting cocaine-induced convulsions (Fisher's exact test, P<0.05 at least one dose). Furthermore, there appears to be a relationship between the length of the substitution and the protective ability, with improved efficacy going from H (BD1060) to methyl (BD1008) to ethyl (BD1067). Analogs with alterations to the pyrrolidinyl ring of BD1008 (BD1047, LR 172) also produced dramatic reduction of cocaine-induced convulsions.

Conformationally-restricted analogs of BD1008 (BD1018, BD1063, LR132, LR176) likewise dramatically attenuated cocaine-induced convulsions, producing significant levels of protection at all doses tested. In addition, there appears to be some stereoselectivity to this response because BD1031, the trans-isomer of BD1018, had agnostic activity and was unable to prevent the convulsive effects of cocaine; this pattern of results is consistent with the agonistic effects of BD1031 in the dystonia studies.

The aryl monosubstituted analogs with high affinity for sigma receptors also attenuated cocaine-induced convulsions. YZ-011, YZ-016, YZ-018, YZ-027, YZ-028, and YZ-029 virtually eliminated cocaine-induced convulsions across a wide dose range (0.01-30 mg/kg, i.p.) Remarkably, there were no obvious behavioral toxic side effects of any of the sigma ligands antagonists tested (e.g. no sedation, ataxia, hyper/hypothermia) and the wide therapeutic range of many of the compounds indicate that they may have a particularly favorable margin of safety.

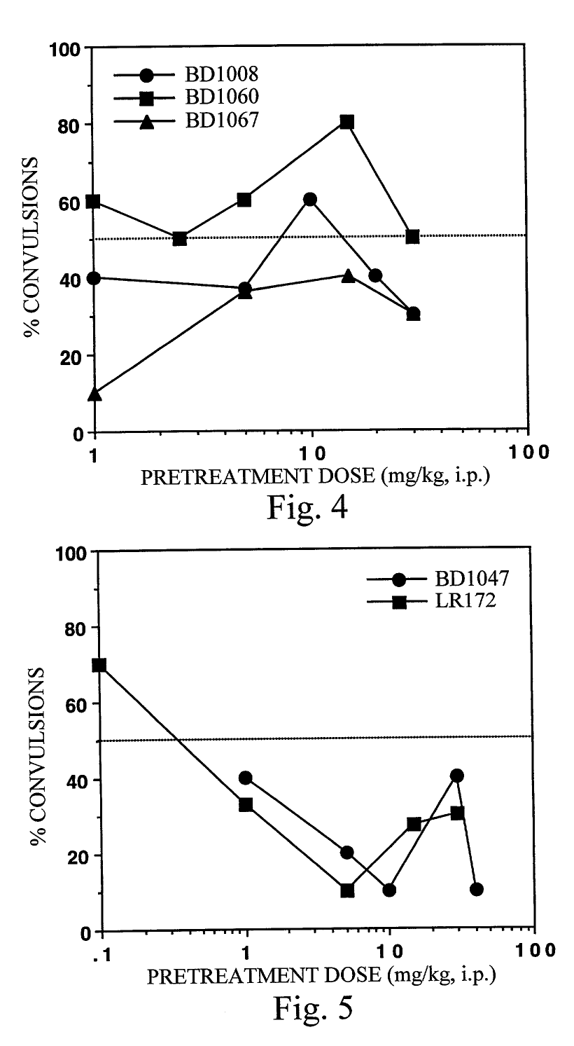

FIG. 4 shows that N-alkyl substituted compounds attenuate cocaine-induced convulsions. Male, Swiss Webster mice were injected (i.p.) with a dose of BD1008, BD1060, or BD1067 (0-30 mg/kg), followed 15 min later with a convulsive dose of cocaine (60 mg/kg, i.p.). The mice were observed continuously for the next 30 min for the onset of convulsions. Pre-treatment of mice with control injections of saline resulted in convulsions in all of the animals. In contrast, Fisher's exact test revealed a significant attenuation in the proportion of mice exhibiting cocaine-induced convulsions when they were pre-treated with BD1008, BD1060, or BD1067 (P<0.05 at least one dose). Values that fall at or below the dotted line at 50% signify statistically significant reductions.

FIG. 5 shows that pyrrolidinyl ring altered compounds attenuate cocaine-induced convulsions. Male, Swiss Webster mice were injected (i.p.) with a dose of BD1047 or LR172 (0-30 mg/kg), followed 15 min later with a convulsive dose of cocaine (60 mg/kg, i.p.). The mice were observed continuously for the next 30 min for the onset of convulsions. Pre-treatment of mice with control injections of saline resulted in convulsions in all of the animals. In contrast, Fisher's exact test revealed a significant attenuation in the proportion of mice exhibiting cocaine-induced convulsions when they were pre-treated with BD1047 or LR172 (P<0.05 at least one dose). Values that fall at or below the dotted line at 50% signify statistically significant reductions.

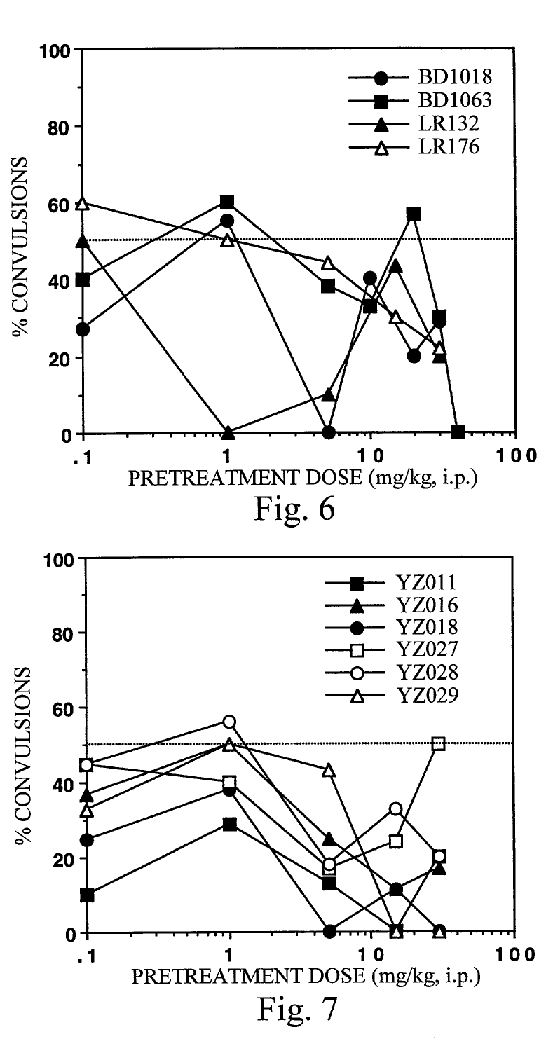

FIG. 6 shows that conformationally-restricted compounds attenuate cocaine-induced convulsions. Male, Swiss Webster mice were injected (i.p.) with a dose of BD1018, BD1063, LR132 or LR176 (0-30 mg/kg), followed 15 min later with a convulsive dose of cocaine (60 mg/kg, i.p.). The mice were observed continuously for the next 30 min for the onset of convulsions. Pre-treatment of mice with control injections of saline resulted in convulsions in all of the animals. In contrast, Fisher's exact test revealed a significant attenuation in the proportion of mice exhibiting cocaine-induced convulsions when they were pre-treated with BD1018, BD1063, LR132 or LR176 (P<0.05 at least one dose). Values that fall at or below the dotted line at 50% signify statistically significant reductions.

FIG. 7 shows that the aryl monosubstituted compounds attenuate cocaine-induced convulsions. Male, Swiss Webster mice were injected (i.p.) with a dose of YZ011, YZ016, YZ018, YZ027, YZ028 or YZ029 (0-30 mg/kg), followed 15 min later with a convulsive dose of cocaine (60 mg/kg, i.p.). The mice were observed continuously for the next 30 min for the onset of convulsions. Pre-treatment of mice with control injections of saline resulted in convulsions in all of the animals. In contrast, Fisher's exact test revealed a significant attenuation in the proportion of mice exhibiting cocaine-induced convulsions when they were pre-treated with YZ011, YZ016, YZ018, YZ027, YZ028 or YZ029 (P<0.05 at least one dose). Values that fall at or below the dotted line at 50% signify statistically significant reductions.

In addition to the core group of compounds described thus far, the second series of novel ligands, listed in Table 4, which have more extensive modifications to the BD1008 structure, also attenuate the convulsive effects of cocaine. Male, Swiss, Webster mice were injected (i.p.) With a dose of YZ-048-2, YZ-051-2, YZ-067-2, YZ-069-2, YZ-085-2, YZ-136-2, YZ-155-2, YZ-165-2-2, YZ-166-2-2, and YZ-184-2 (0-30 mg/kg), followed 15 minutes later with a convulsive dose of cocaine (60 mg/kg i.p.). The mice were observed continuously for the next 30 minutes for the onset of convulsions. Fisher's exact test revealed that all of the compounds from this group significantly attenuated cocaine-induced convulsions.

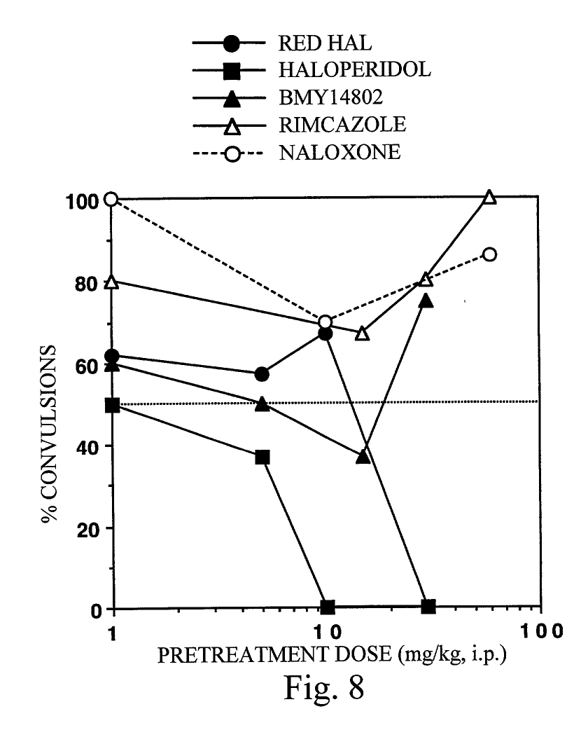

The “historic sigma” compounds were tested to serve as a reference against which the effects of the novel ligands could be compared. As shown in FIG. 8, the “historic sigma” compounds vary from non-sigma affinity to relatively high affinity. Male, Swiss Webster mice were injected (i.p.) with a dose (0-60 mg/kg) of haloperidol, reduced haloperidol, BMY14802, rimcazole, or naloxone, followed 15 min later with a convulsive dose of cocaine (60 mg/kg, i.p.). The mice were observed continuously for the next 30 min for the onset of convulsions. Pre-treatment of mice with control injections of saline resulted in convulsions in all of the animals. In contrast, Fisher's exact test revealed a significant attenuation in the proportion of mice exhibiting cocaine-induced convulsions when they were pre-treated with the high affinity sigma ligands haloperidol or reduced haloperidol (P<0.05 at least one dose, values that fall at or below the dotted line at 50% signify statistically significant reductions.). However, at these therapeutic doses, haloperidol and reduced haloperidol produced marked sedation in the animals. The moderate affinity sigma ligand BMY14802 also significantly attenuated cocaine-induced convulsions (P<0.05) although it had a reduced maximal effect as compared to the high affinity compounds. The historic “sigma receptor antagonist” rimcazole failed to produce significant protection against cocaine-induced convulsions, a pattern that is consistent with its low affinity for sigma receptors. The opiate antagonist naloxone, which lacks affinity for sigma receptors, also failed to attenuated the convulsive effects of cocaine. Therefore the “historic sigma” ligands were either less effective or associated with pronounced side effects as compared to the novel compounds.

If sigma receptor antagonists attenuate the convulsive effects of cocaine, then sigma receptor agonists should worsen the toxicity (shown in FIG. 9). Sigma receptor agonists indeed worsen the convulsive effects of cocaine. Male, Swiss Webster mice were injected (i.p.) with saline or a 30 mg/kg, i.p. dose of a traditional sigma receptor agonist (DTG, (+)-pentazocine), or a novel sigma receptor agonist (BD1052, BD1031), followed 15 min later with a dose of cocaine (0-60 mg/kg, i.p.). The mice were observed continuously for the next 30 min for the onset of convulsions. None of the sigma receptor agonists prevented the convulsive effects of cocaine. DTG, BD1052 and BD1031 in fact worsened the toxic effects of cocaine, producing a shift to the left in the dose curve for cocaine. The ED₅₀ for cocaine-induced convulsions shifted from 58 mg/kg, i.p. to 33 mg/kg, i.p. in the presence of DTG, to 46 mg/kg, i.p. in the presence of BD1052, and to 47 mg/kg, i.p. in the presence of BD103 1. (+)-Pentazocine, which is often considered a selective σ₁ receptor agonist, failed to shift the dose curve for cocaine-induced convulsions. This apparent inconsistency with (+)-pentazocine may be related to its known ability to interact with additional non-σ₁/σ₂ sites under in vivo conditions or its binding to a different position on the receptor complex from DTG.

The ability of the sigma ligands described and claimed herein to prevent cocaine-induced lethality was also tested since it is the ultimate toxic endpoint.

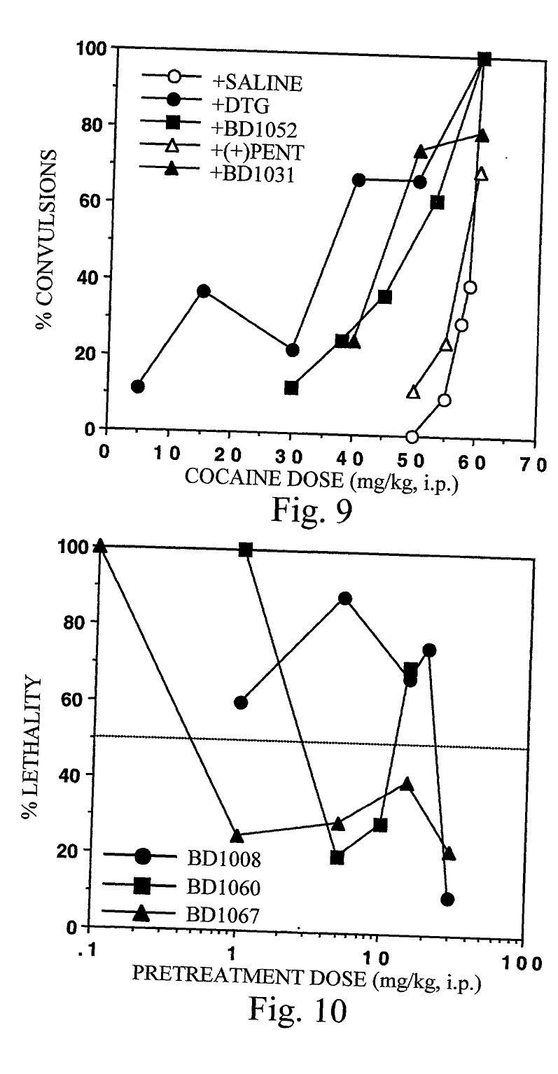

As shown in FIG. 10, the N-alkyl substituted compounds attenuate cocaine-induced lethality. Male, Swiss Webster mice were injected (i.p.) with a dose of BD1008, BD1060, or BD1067 (0-30 mg/kg), followed 15 min later with a lethal dose of cocaine (125 mg/kg, i.p.). The mice were observed continuously for the next 30 min for death. Pre-treatment of mice with control injections of saline resulted in the death of all animals. In contrast, Fisher's exact test revealed a significant attenuation in the proportion of mice exhibiting cocaine-induced lethality when they were pre-treated with BD1008, BD1060, or BD1067 (P<0.05 at least one dose); Values that fall at or below the dotted line at 50% signify statistically significant reductions.

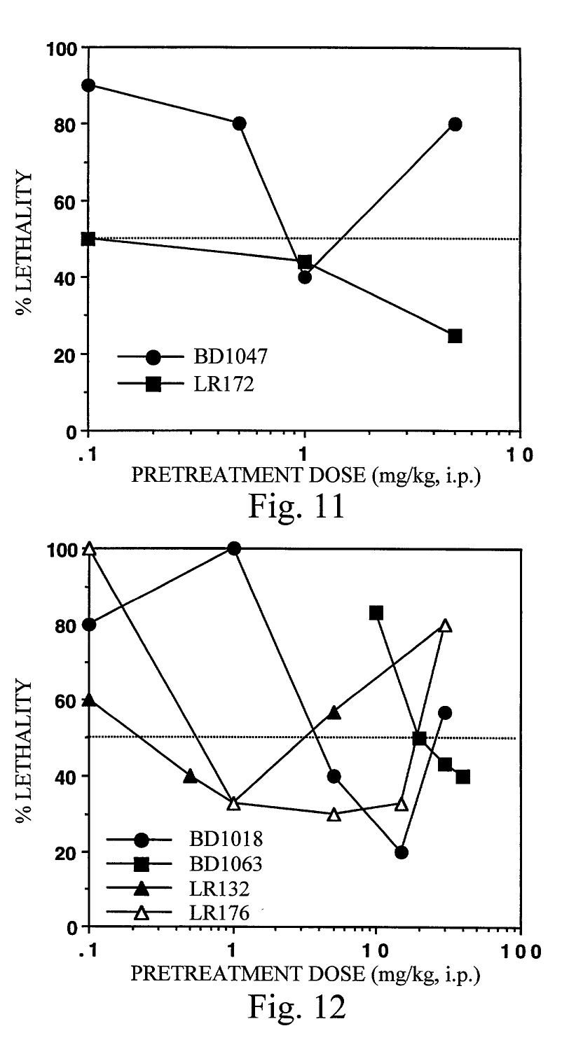

FIG. 11 shows that pyrrolidinyl ring altered compounds also attenuate cocaine-induced lethality. Male, Swiss Webster mice were injected (i.p.) with a dose of BD1047 or LR172 (0-30 mg/kg), followed 15 min later with a lethal dose of cocaine (125 mg/kg, i.p.). The mice were observed continuously for the next 30 min for death. Pre-treatment of mice with control injections of saline resulted in the death of all animals. In contrast, Fisher's exact test revealed a significant attenuation in the proportion of mice exhibiting cocaine-induced lethality when they were pre-treated with BD1047 or LR172 (P<0.05 at least one dose; Values that fall at or below the dotted line at 50% signify statistically significant reductions).

As shown in FIG. 12, conformationally-restricted compounds attenuate cocaine-induced lethality. Male, Swiss Webster mice were injected (i.p.) with a dose of BD1018, BD1063, LR132 or LR176 (0-30 mg/kg), followed 15 min later with a lethal dose of cocaine (125 mg/kg, i.p.). The mice were observed continuously for the next 30 min for death. Pre-treatment of mice with control injections of saline resulted in the death of all animals. In contrast, Fisher's exact test revealed a significant attenuation in the proportion of mice exhibiting cocaine-induced lethality when they were pre-treated with BD1018, BD1063, LR132 or LR176 (P<0.05 at least one dose; Values that fall at or below the dotted line at 50% signify statistically significant reductions).

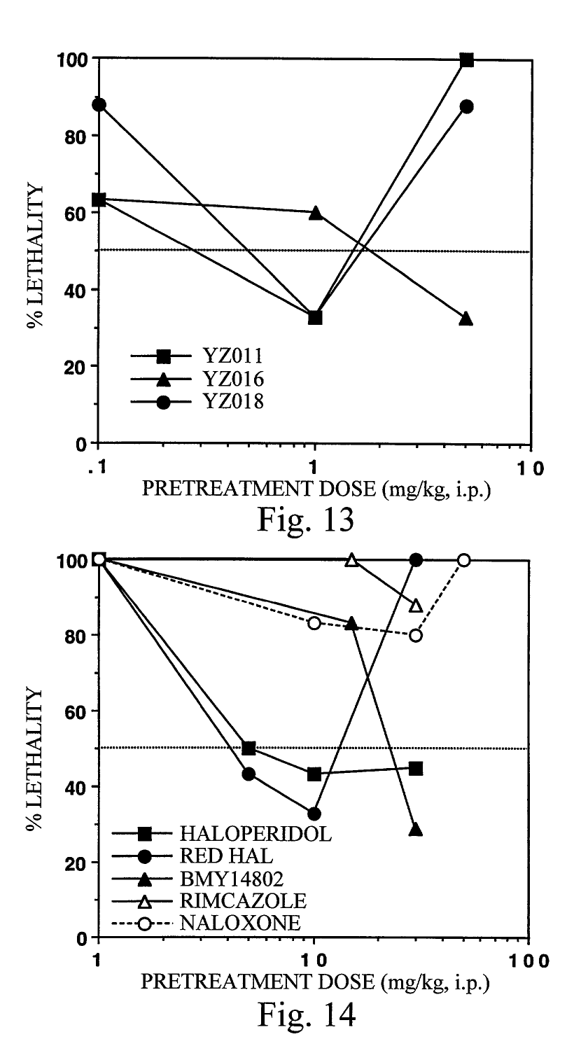

Likewise, FIG. 13 shows that the aryl monosubstituted compounds attenuate cocaine-induced lethality. Male, Swiss Webster mice were injected (i.p.) with a dose of YZ011, YZ016, or YZ018 (0-5 mg/kg), followed 15 min later with a lethal dose of cocaine (125 mg/kg, i.p.). The mice were observed continuously for the next 30 min for death. Pre-treatment of mice with control injections of saline resulted in the death of all animals. In contrast, Fisher's exact test revealed a significant attenuation in the proportion of mice exhibiting cocaine-induced lethality when they were pre-treated with YZ011, YZ016, or YZ018 (P<0.05 at least one dose; Values that fall at or below the dotted line at 50% signify statistically significant reductions).

As with convulsions, the “historic sigma” compounds vary in their ability to attenuate cocaine-induced lethality, as shown in FIG. 14. Male, Swiss Webster mice were injected (i.p.) with a dose (0-60 mg/kg) of haloperidol, reduced haloperidol, BMY14802, rimcazole, or naloxone, followed 15 min later with a lethal dose of cocaine (125 mg/kg, i.p.). The mice were observed continuously for the next 30 min for death. Pre-treatment of mice with control injections of saline resulted in the death of all animals. In contrast, Fisher's exact test revealed a significant attenuation in the proportion of mice exhibiting cocaine-induced lethality when they were pre-treated with the high or moderate affinity sigma ligands haloperidol, reduced haloperidol, or BMY14802 (P<0.05 at least one dose; Values that fall at or below the dotted line at 50% signify statistically significant reductions). Similar to the pattern observed in the convulsion studies, the low affinity “sigma receptor antagonist” rimcazole and the opiate antagonist naloxone were ineffective.

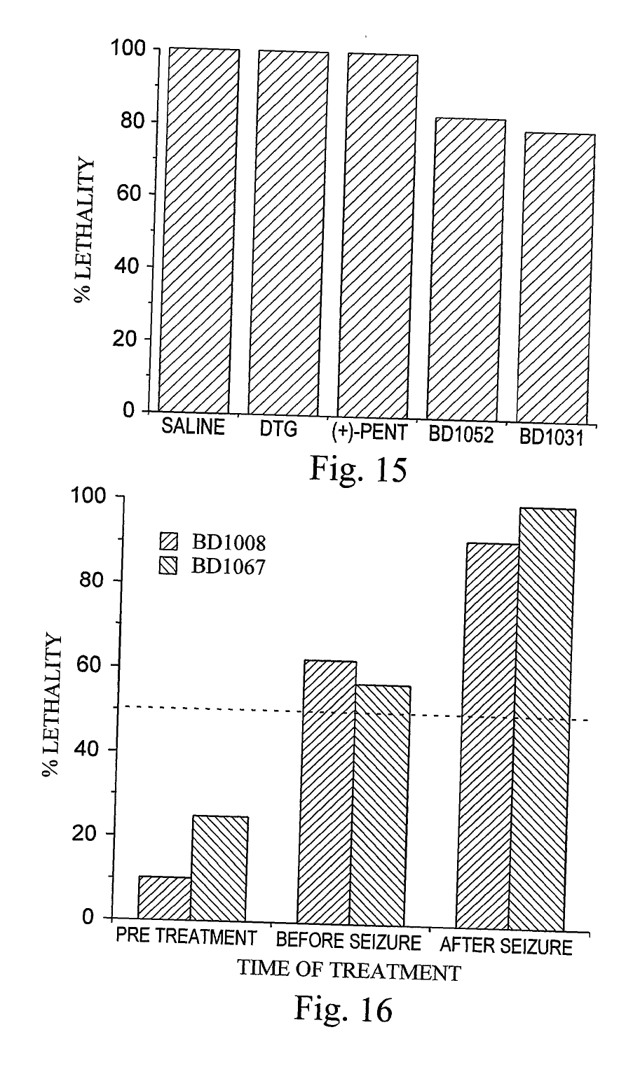

Also, as shown in FIG. 15, the sigma receptor agonists fail to attenuate cocaine-induced lethality. Male, Swiss Webster mice were injected (i.p.) with saline or a 30 mg/kg, i.p. dose of a traditional sigma receptor agonist (DTG, (+)-pentazocine), or a novel sigma receptor agonist (BD1052, BD1031), followed 15 min later with a lethal dose of cocaine (125 mg/kg, i.p.). The mice were observed continuously for the next 30 min for death. Similar to the pattern observed in the convulsion experiments, the sigma receptor agonists DTG, (+)-pentazocine, BD1052 and BD1031 failed to protect against the lethal effects of cocaine.

In the majority of lethality studies, the sigma ligands were administered as a pretreatment to ensure that the receptors were occupied at the time of overdose. To be of clinical use, however, the compounds must be effective when administered after cocaine. Therefore, a select group of ligands were tested for their ability to attenuate death when administered after a normally lethal dose of cocaine. Male, Swiss Webster, mice were administered a normally lethal dose of cocaine (125 mg/kg, i.p.). Following the administration of this lethal dose of cocaine, the mice typically begin convulsing after approximately 2 minutes and are dead at about 4.5 minutes. The mice were administered a sigma ligand (i.p.) Either just prior to or just after the onset of convulsions, thereby allowing about 2.5 minutes in which to reverse the toxicity. The mice were observed for the next 30 min, and checked again after 24 hours, for death. The compounds that were tested either attenuated the lethal effects of cocaine when administered as a post-treatment, or had marginal effects. Those that produced marginal effects under the post-treatment conditions, exhibited a trend of increased efficacy the earlier they were administered, suggesting that alternate doses or routes of administration to increase absorption or drug levels at relevant time points would improve their clinical relevance.

FIG. 16 shows that the post-treatment with N-alkyl substituted compound did not attenuate cocaine-induced lethality. Male, Swiss Webster mice were injected with a lethal dose of cocaine (125 mg/kg, i.p.). The mice were then post-treated with a dose of BD1008, (30 mg/kg, i.p.) or BD1067 (1 mg/kg, i.p.) that effectively prevented cocaine-induced lethality when administered as a pretreatment; similar injections of saline served as the control. Separate groups of mice received the post-treatments either immediately before or immediately after the onset of convulsions. Following the administration of a lethal dose of cocaine, the mice typically begin convulsing after about 2 min and are dead at about 4.5 min, thereby allowing a 2.5 min therapeutic window for the post-treatments. Control injections of saline resulted in cocaine-induced lethality of all mice regardless of the time point at which it was administered (pretreatment, before post treatment seizure, after post treatment seizure). Post-treatment with BD1008 or BD1067 under the conditions of these experiments did not provide significant protection against the lethal effects of cocaine. There was a trend however for improved protective ability when the compounds were administered earlier. Therefore, other routes of administration (e.g. i.v.) to improve absorption or other doses of the compounds may improve the therapeutic effects of the ligands when administered as a post-treatment.

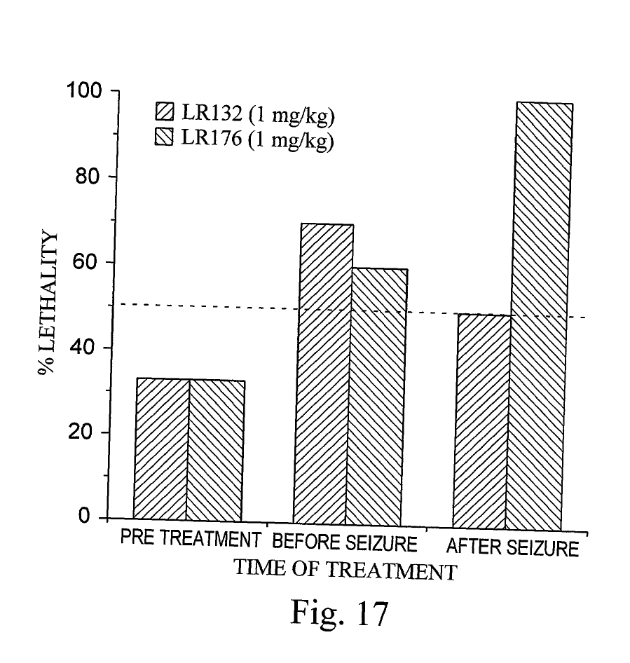

FIG. 17, whereas, shows that post-treatment with conformationally restricted compounds varied in their ability to attenuate cocaine-induced lethality. Male, Swiss Webster mice were injected with a lethal dose of cocaine (125 mg/kg, i.p.). The mice were then post-treated with doses of LR132 (0.1, 1 mg/kg, i.p.) or LR176 (1, 5 mg/kg, i.p.) that effectively prevented cocaine-induced lethality when administered as a pretreatment; similar injections of saline served as the control. Separate groups of mice received the post-treatments either immediately before or immediately after the onset of convulsions. Following the administration of a lethal dose of cocaine, the mice typically begin convulsing after about 2 min and are dead at about 4.5 min, thereby allowing a 2.5 min therapeutic window for the post-treatments. Control injections of saline resulted in cocaine-induced lethality of all mice regardless of the time point at which it was administered (pre, before seizure, after seizure). Post-treatment with LR132 provided significant protection against the lethal effects of cocaine. Similar to many of the other compounds tested under the post-treatment conditions, LR176 failed to prevent death in a significant proportion of animals although there was a trend for improved protective ability when it was administered earlier.

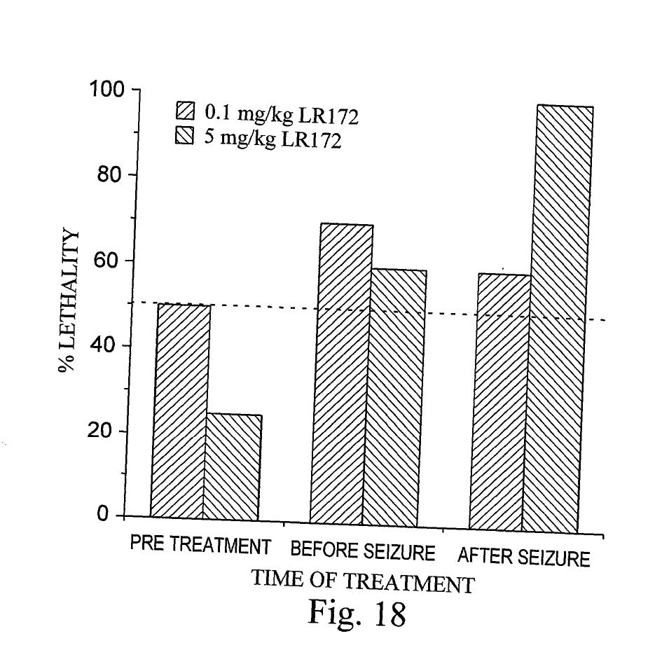

FIG. 18 shows that post-treatment with a pyrrolidinyl ring altered compound did not attenuate cocaine-induced lethality. Male, Swiss Webster mice were injected with a lethal dose of cocaine (125 mg/kg, i.p.). The mice were then post-treated with doses of LR172 (0.1, 5 mg/kg, i.p.) that effectively prevented cocaine-induced lethality when administered as a pretreatment; similar injections of saline served as the control. Separate groups of mice received the post-treatments either immediately before or immediately after the onset of convulsions. Following the administration of a lethal dose of cocaine, the mice typically begin convulsing after about 2 min and are dead at about 4.5 min, thereby allowing a 2.5 min therapeutic window for the post-treatments. Control injections of saline resulted in cocaine-induced lethality of all mice regardless of the time point at which it was administered (pre, before seizure, after seizure). Post-treatment with LR172 under the conditions of these experiments did not provide significant protection against the lethal effects of cocaine. There was a trend however for improved protective ability when the compound was administered earlier. Therefore, other routes of administration (e.g. i.v.) to improve absorption or other doses of the compounds may improve the therapeutic effects of LR172 when administered as a post-treatment.

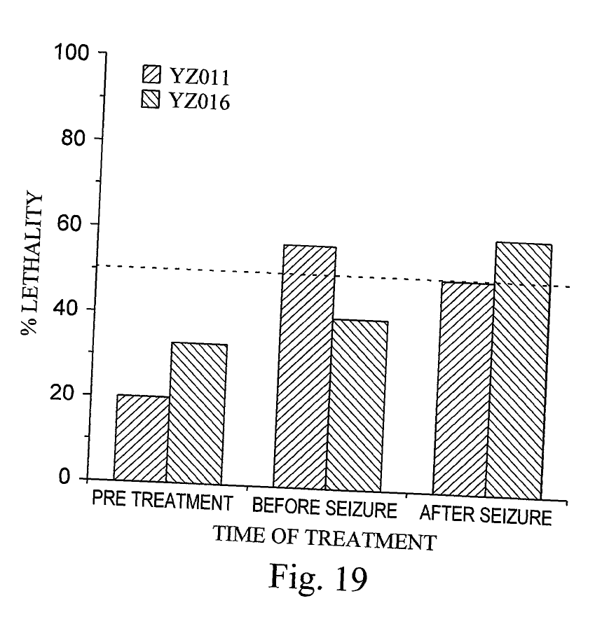

Finally, FIG. 19 shows that post-treatment with aryl monosubstituted compounds attenuated cocaine-induced lethality. Male, Swiss Webster mice were injected with a lethal dose of cocaine (125 mg/kg, i.p.). The mice were then post-treated with a dose of YZ11 (1 mg/kg, i.p.) or YZ016 (5 mg/kg, i.p.) that effectively prevented cocaine-induced lethality when administered as a pretreatment; similar injections of saline served as the control. Separate groups of mice received the post-treatments either immediately before or immediately after the onset of convulsions. Following the administration of a lethal dose of cocaine, the mice typically begin convulsing after about 2 min and are dead at about 4.5 min, thereby allowing a 2.5 min therapeutic window for the post-treatments. Control injections of saline resulted in cocaine-induced lethality of all mice regardless of the time point at which it was administered (pre, before seizure, after seizure). Post-treatment with YZ011 or YZ016 provided significant protection against the lethal effects of cocaine (P<0.05 for at least one post-treatment condition).

The results from the convulsion and lethality experiments demonstrate that the sigma receptor antagonists described and claimed herein have powerful anti-cocaine actions. Therefore, the ability of the sigma receptor antagonists described and claimed herein were experimented as to their effects on psychomotor stimulatory actions of cocaine. The actions of the sigma ligands on locomotor activity were first tested alone then in combination with cocaine.

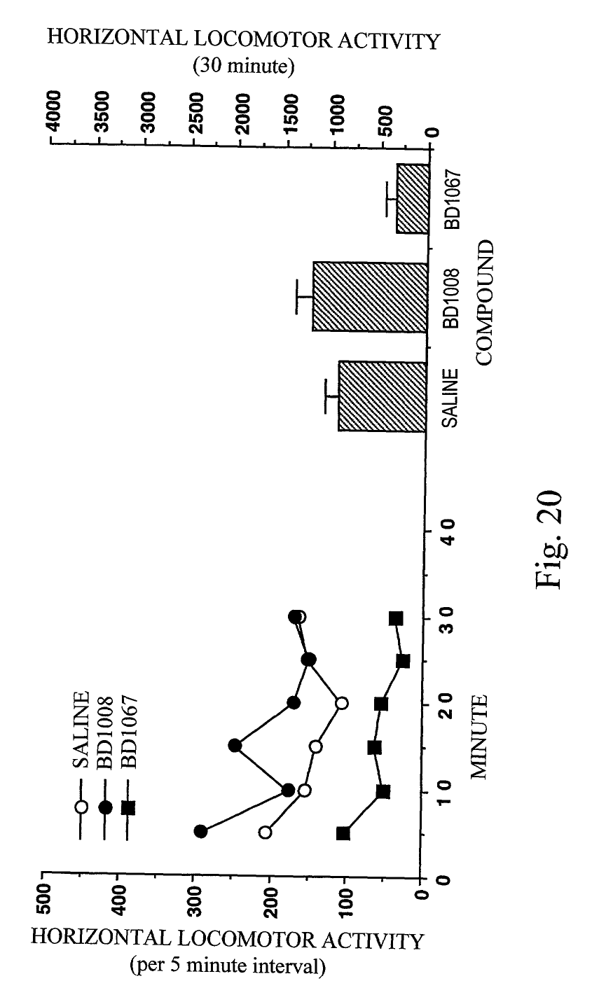

FIG. 20 shows the effects of N-alkyl substituted ligands on baseline locomotor activity. Male, Swiss Webster mice were acclimated for 30 min to the plexiglas enclosures of an automated activity monitor (San Diego Instruments, San Diego, Calif.). The mice were then injected with saline, BD1008 (1 mg/kg, i.p.) or BD1067 (5 mg/kg, i.p.) and horizontal locomotor activity was quantified for the subsequent 30 min as the number of disruptions in the 4×4 photobeam array that surrounded each enclosure. Analysis of variance revealed no significant difference in the level of locomotor activity produced by BD1008 and saline, although there was a significant reduction in locomotor activity in response to this dose of BD1067 (P<0.05).

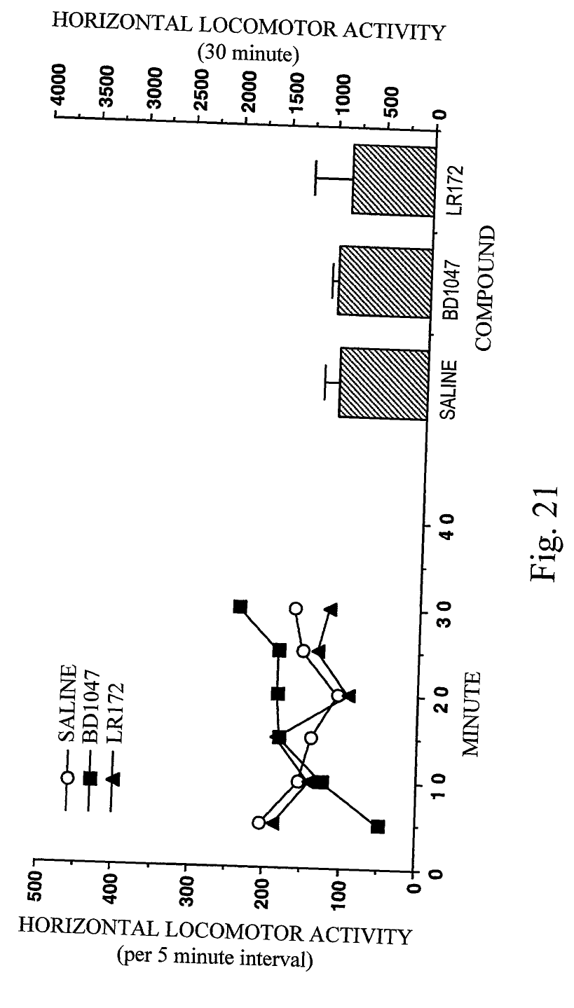

FIG. 21 shows the effects of pyrrolidinyl ring altered ligands on baseline locomotor activity. Male, Swiss Webster mice were acclimated for 30 min to the plexiglas enclosures of an automated activity monitor (San Diego Instruments, San Diego, Calif.). The mice were then injected with saline, BD1047 (30 mg/kg, i.p.) or LR172 (5 mg/kg, i.p.) and horizontal locomotor activity was quantified for the subsequent 30 min as the number of disruptions in the 4×4 photobeam array that surrounded each enclosure. Analysis of variance revealed no significant difference in the level of locomotor activity produced by saline, BD1047 and LR172.

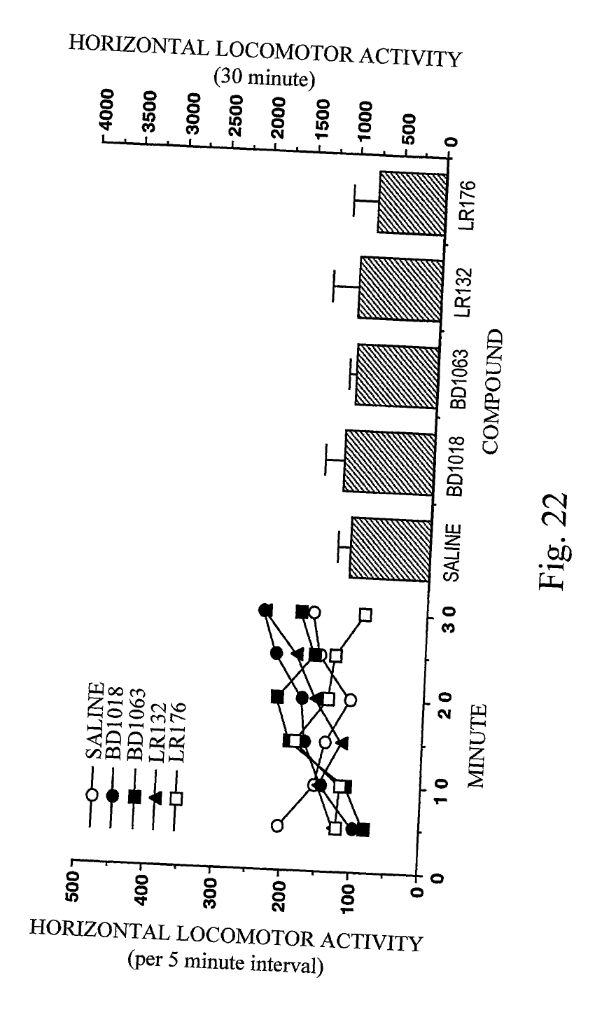

FIG. 22 shows the effects of conformationally restricted ligands on baseline locomotor activity. Male, Swiss Webster mice were acclimated for 30 min to the plexiglas enclosures of an automated activity monitor (San Diego Instruments, San Diego, Calif.). The mice were then injected with saline, BD1018 (30 mg/kg, i.p.), BD1063 (30 mg/kg, i.p.), LR132 (30 mg/kg, i.p.) or LR 76 (10 mg/kg, i.p.) and horizontal locomotor activity was quantified for the subsequent 30 min as the number of disruptions in the 4×4 photobeam array that surrounded each enclosure. Analysis of variance revealed no significant difference in the level of locomotor activity produced by saline, BD1018, BD1063, LR132 and LR176.

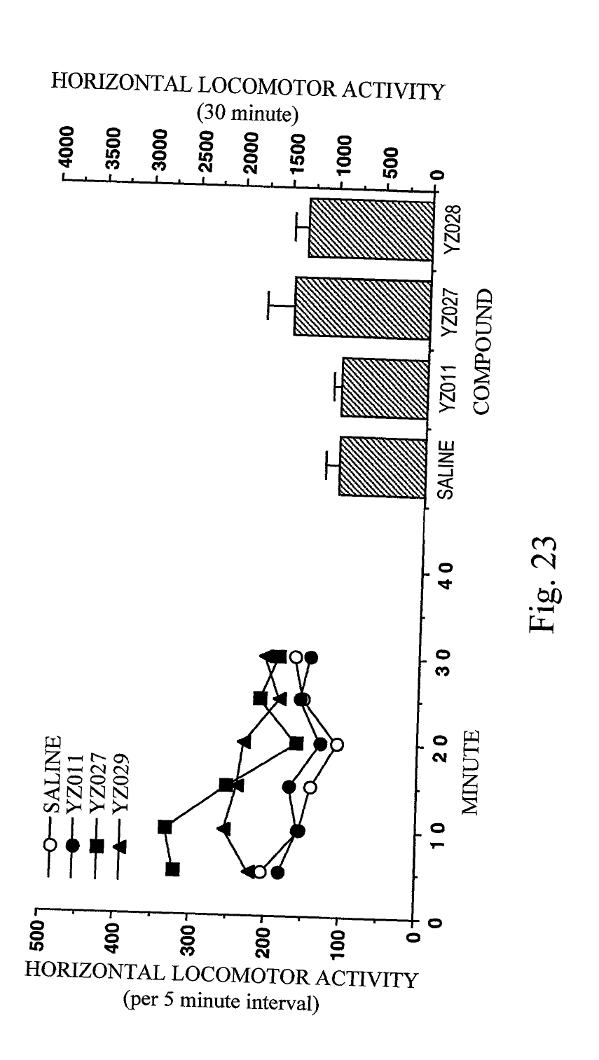

FIG. 23 shows the effects of aryl monosubstituted ligands on baseline locomotor activity. Male, Swiss Webster mice were acclimated for 30 min to the plexiglas enclosures of an automated activity monitor (San Diego Instruments, San Diego, Calif.). The mice were then injected with saline, YZ011 (0.1 mg/kg, i.p.), YZ027 (1 mg/kg, i.p.) or YZ029 (5 mg/kg, i.p.) and horizontal locomotor activity was quantified for the subsequent 30 min as the number of disruptions in the 4×4 photobeam array that surrounded each enclosure. Analysis of variance revealed no significant difference in the level of locomotor activity produced by saline, YZ011, YZ027 and YZ029.

These doses of sigma ligands were then tested in combination with cocaine. FIG. 3 shows the dose response for the locomotor stimulatory effects of cocaine alone. Male, Swiss Webster mice were acclimated for 30 min to the plexiglass enclosures of an automated activity monitor (San Diego Instruments, San Diego, Calif.). The mice were then injected (i.p.) with a dose of cocaine (0-20 mg/kg) and horizontal locomotor activity was quantified for the next 30 min by the computer as the number of disruptions in the 4×4 photobeam array that surrounded each plexiglas enclosure. The cocaine produced a dose-dependent increase in the level of locomotor activity (F[4,20]=41.58, P<0.0001). Post-hoc Dunnett's tests confirmed a significant difference between the saline control and each of the cocaine doses (P<0.01). The dose of cocaine that produced the peak level of locomotor activity (10 mg/kg, i.p.) was selected as the challenge dose in the antagonism experiments detailed below.

At doses where they produced no significant effect on locomotor activity (excepting BD1067 which has a locomotor depressant effect), all of the sigma ligands described and claimed herein attenuated the locomotor stimulatory effects of cocaine.

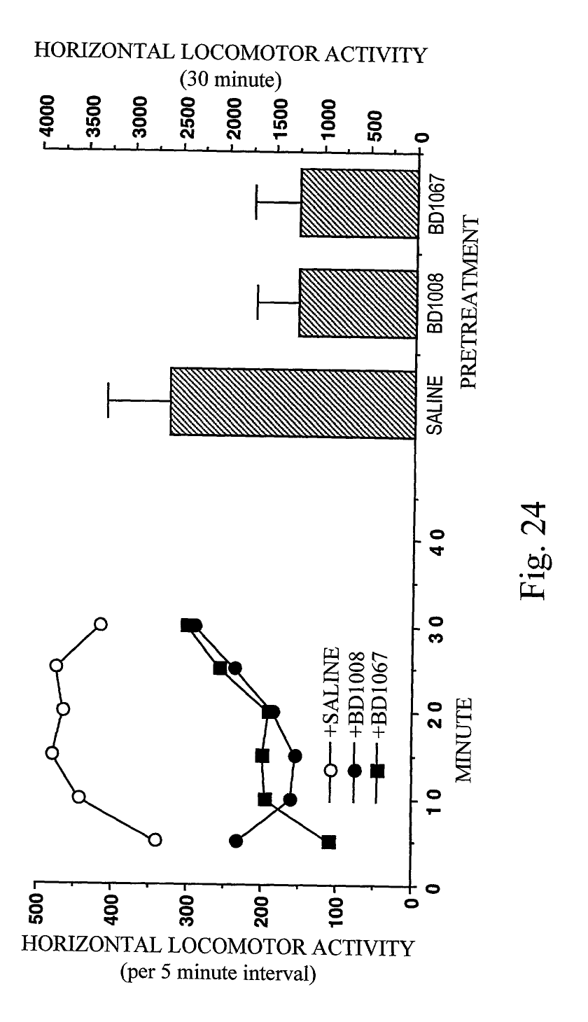

FIG. 24 shows that N-alkyl substituted ligands attenuate cocaine-induced locomotor activity. Male, Swiss Webster mice were acclimated for 15 min to the plexiglas enclosures of an automated activity monitor (San Diego Instruments, San Diego, Calif.). The mice were then injected with saline, BD1008 (1 mg/kg, i.p.) or BD1067 (5 mg/kg, i.p.). After a 15 min pretreatment period (total 30 min acclimation period), the mice were injected with a locomotor stimulatory dose of cocaine (10 mg/kg, i.p.). Horizontal locomotor activity was quantified for the subsequent 30 min as the number of disruptions in the 4×4 photobeam array that surrounded each enclosure. Mice that were pretreated with BD1008 or BD1067 exhibited a significant reduction in cocaine stimulated locomotor activity. Analysis of variance confirmed that this difference was statistically significant (P<0.05).

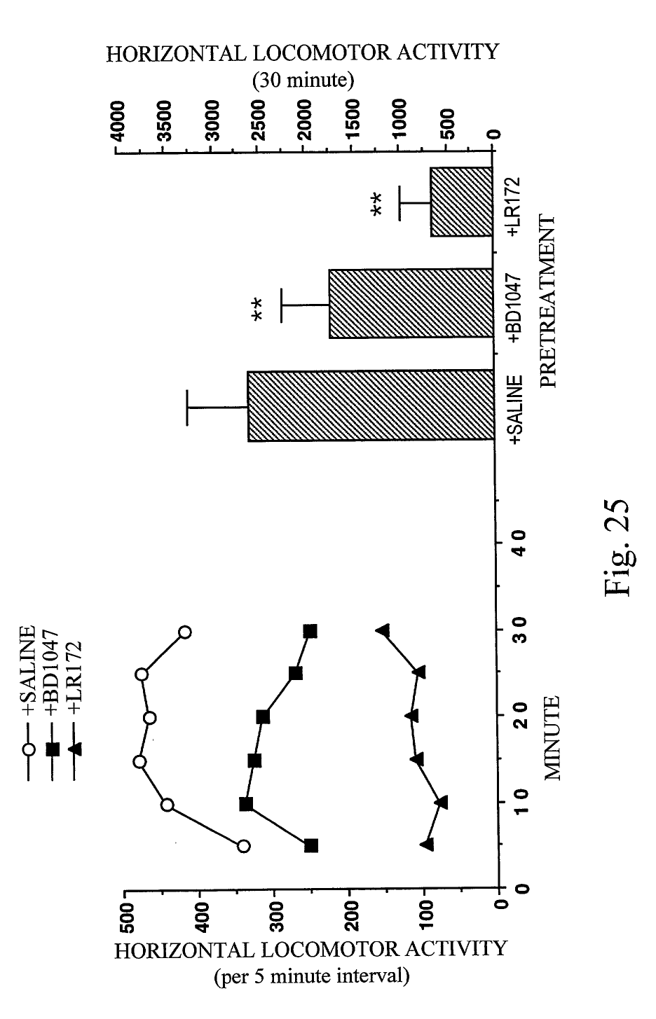

FIG. 25 also shows that pyrrolidinyl ring altered ligands attenuate cocaine-induced locomotor activity. Male, Swiss Webster mice were acclimated for 15 min to the plexiglas enclosures of an automated activity monitor (San Diego Instruments, San Diego, Calif.). The mice were then injected with saline, BD1047 (30 mg/kg, i.p.) or LR172 (5 mg/kg, i.p.), doses previously shown to produce effects no different from saline when administered alone. After a 15 min pretreatment period (total 30 min acclimation period), the mice were injected with a locomotor stimulatory dose of cocaine (10 mg/kg, i.p.). Horizontal locomotor activity was quantified for the subsequent 30 min as the number of disruptions in the 4×4 photobeam array that surrounded each enclosure. Mice that were pretreated with BD1047 or LR172 exhibited a significant reduction in cocaine stimulated locomotor activity. Analysis of variance confirmed that this difference was statistically significant (P<0.05).

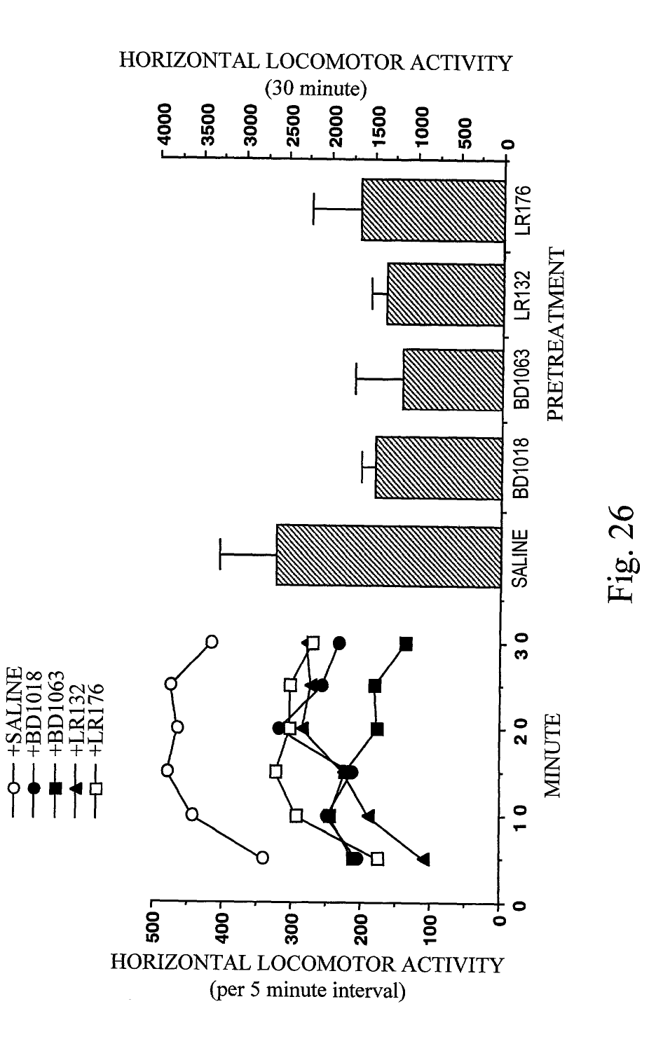

FIG. 26 shows that conformationally restricted ligands attenuate cocaine-induced locomotor activity. Male, Swiss Webster mice were acclimated for 15 min to the plexiglas enclosures of an automated activity monitor (San Diego Instruments, San Diego, Calif.). The mice were then injected with saline, BD1018 (30 mg/kg, i.p.), BD1063 (30 mg/kg, i.p.), LR132 (30 mg/kg, i.p.) or LR176 (10 mg/kg, i.p.), doses previously shown to produce effects no different from saline when administered alone. After a 15 min pretreatment period (total 30 min acclimation period), the mice were injected with a locomotor stimulatory dose of cocaine (10 mg/kg, i.p.). Horizontal locomotor activity was quantified for the subsequent 30 min as the number of disruptions in the 4×4 photobeam array that surrounded each enclosure. Mice that were pretreated with BD1018, BD1063, LR132 or LR176 exhibited a significant reduction in cocaine stimulated locomotor activity. Analysis of variance confirmed that this difference was statistically significant (P<0.05).

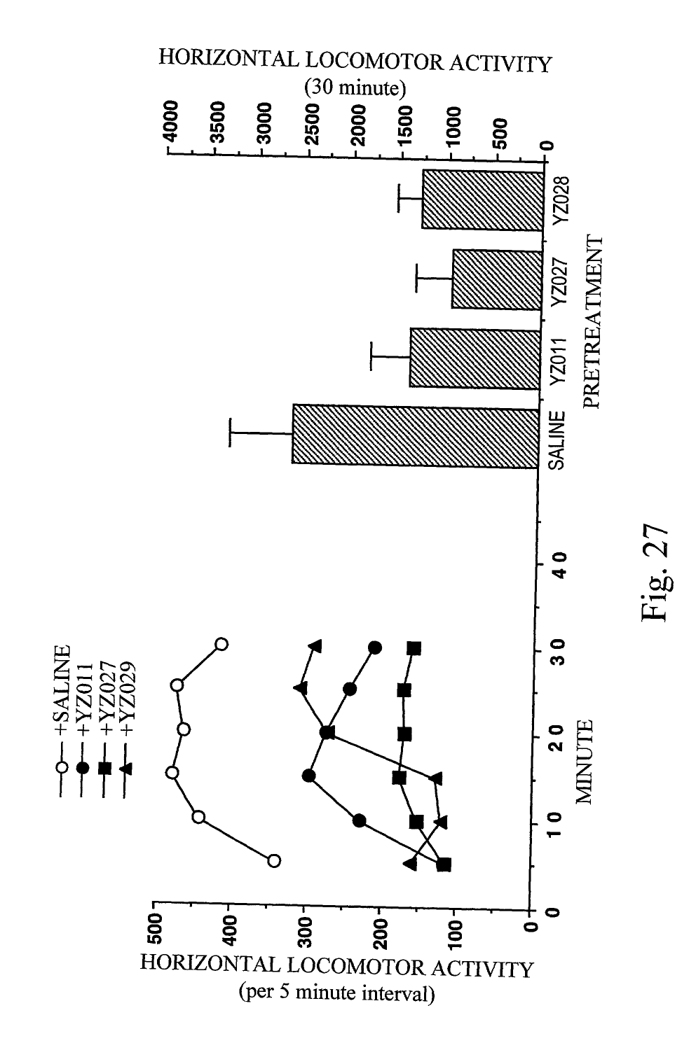

FIG. 27 also shows that aryl monosubstituted ligands attenuate cocaine-induced locomotor activity. Male, Swiss Webster mice were acclimated for 15 min to the plexiglas enclosures of an automated activity monitor (San Diego Instruments, San Diego, Calif.). The mice were then injected with saline, YZ011(0.1 mg/kg, i.p.), YZ027 (1 mg/kg, i.p.) or YZ029 (5 mg/kg, i.p.), doses previously shown to produce effects no different from saline when administered alone. After a 15 min pretreatment period (total 30 min acclimation period), the mice were injected with a locomotor stimulatory dose of cocaine (10 mg/kg, i.p.). Horizontal locomotor activity was quantified for the subsequent 30 min as the number of disruptions in the 4×4 photobeam array that surrounded each enclosure. Mice that were pretreated with YZ011, YZ027 or YZ029 exhibited a significant reduction in cocaine stimulated locomotor activity. Analysis of variance confirmed that this difference was statistically significant (P<0.05).

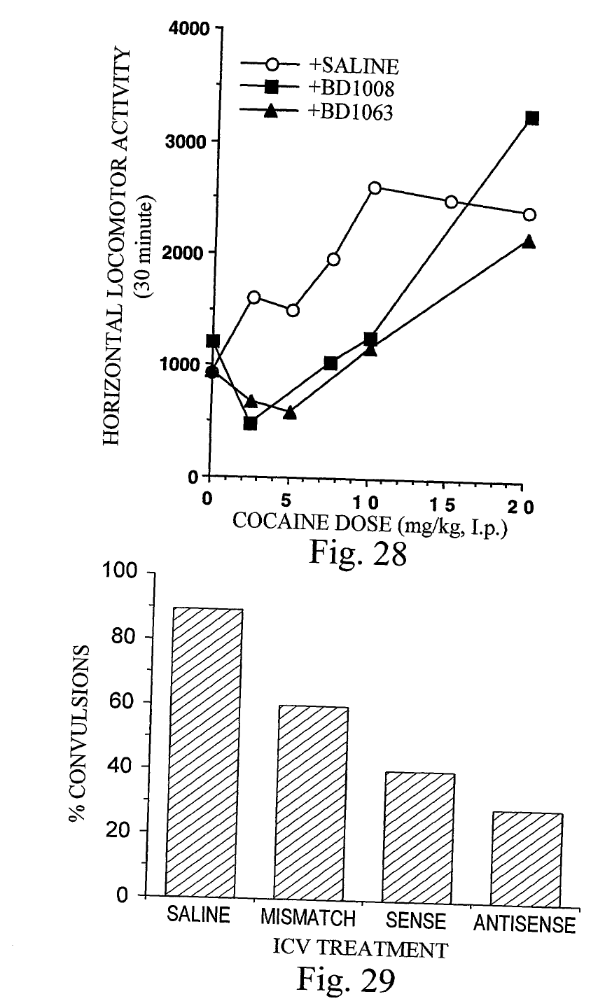

To further validate the actions of the sigma ligands described and claimed herein against the locomotor stimulatory effects of cocaine, two of the sigma ligands described and claimed herein (BD1008, BD1063) were tested against a full dose range of cocaine to determine whether they could also shift the dose curve for the locomotor stimulatory effects of cocaine to the right. As shown in FIG. 28, male, Swiss Webster mice were acclimated for 15 min to the plexiglas enclosures of an automated activity monitor (San Diego Instruments, San Diego, Calif.). The mice were then injected with saline, BD1008 (1 mg/kg, i.p.) or BD1063 (30 mg/kg, i.p.), doses previously shown to produce effects no different from saline when administered alone. After a 15 min pretreatment period (total 30 min acclimation period), the mice were injected with cocaine (0-20 mg/kg, i.p.) and horizontal locomotor activity was quantified for the subsequent 30 min as the number of disruptions in the 4×4 photobeam array that surrounded each enclosure. As illustrated in FIG. 28, the ED₅₀ for the locomotor stimulatory effects of cocaine shifted from 6 mg/kg, i.p. to 11 mg/kg, i.p. in the presence of BD1008 (1 mg/kg, i.p.), and 16 mg/kg, i.p. in the presence of BD1063 (30 mg/kg, i.p.).

In order to provide converging evidence that antagonism of sigma receptors attenuates cocaine-induced behaviors, antisense oligodeoxynucleotides against σ₁ sites were administered to mice i.c.v. that were subsequently challenged with convulsive or locomotor stimulatory dosages of cocaine.

Shown in FIG. 29 is that the NYU antisense oligo against σ₁ receptors attenuates cocaine-induced convulsions. This antisense was described by King M, Pan Y-X, Mei J, Chang A, Xu J, Pasternak G W (1997) Enhanced κ-opioid receptor-mediated analgesia by antisense targeting the σ₁ receptor, Eur J Pharmacol 331:R5-6. It is a 21 -mer that targets area −97 to −77 after the initiation codon of a cloned cDNA sequence for σ₁ receptors from mouse: 5′-GAGTGCCCAGCCACAACCAGG-3′. As a control, three base pairs in the antisense sequence were reversed to obtain the following mismatch sequence: 5′-GAGGTCCCGACCACACACAGG-3′. A sense sequence was also used. The oligodeoxynucleotides were synthesized with a phosphorothioate backbone using an Applied Biosystems 394 DNA Sequencer and purified using HPLC (Molecular Biology Resource Facility, University of Oklahoma Health Sciences Center, Oklahoma City, Okla.).

In order to administer the oligodeoxynucleotides, Male, Swiss Webster mice were first surgically implanted with chronic, indwelling guide cannula with their tips in the left lateral ventricle. To knock down sigma receptors, a total of three intracerebroventricular infusions (each 10 μg/5 μl) of the antisense oligodeoxynucleotide were administered on Days 1, 2 and 4 (King et al., 1997). As controls, a mismatch sequence, the sense sequence, or saline vehicle was administered using the same dosing schedule. On Day 5, the mice were challenged with a convulsive dose of cocaine (60 mg/kg, i.p.) and observed for the next 30 min for the onset of convulsions. After behavioral testing, the intracerebroventricular injection sites were confirmed histologically and the brains were frozen for later confirmation of receptor knockdown.

Fisher's exact test confirmed that prior administration of the NYU antisense protected against the convulsive effects of cocaine (P<0.05). Although statistically, mice treated with mismatch or sense sequences exhibited responses no different from the saline-treated animals in response to cocaine, there was a noticeable attenuation, suggesting that the “control” oligodeoxynucleotides may have some non-specific actions.

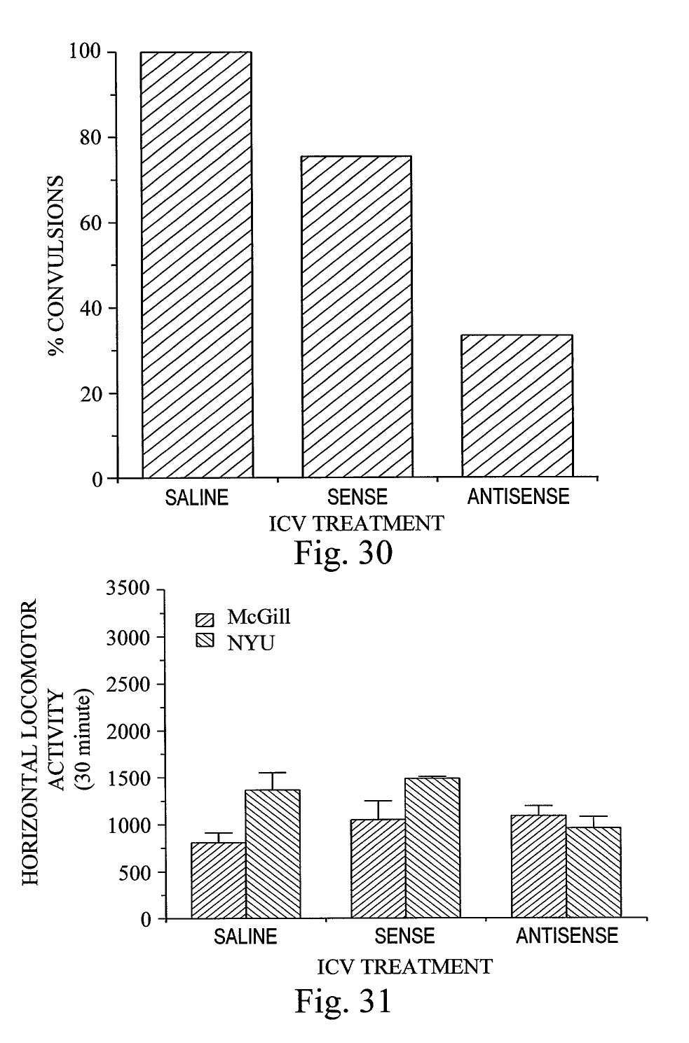

FIG. 30 shows that the McGill antisense oligodeoxynucleotide against σ₁ receptors attenuate cocaine-induced convulsions. This antisense was described by Kitaich K, Chabot J-G, Dumont Y, Bouchard P, Quirion R (1997) Antisense oligodeoxynucleotide against the sigma, receptor regulates MK-801 -induced memory deficits in mice, Soc Neurosci Abst 23:695.23. It is an 18-mer that targets the 5′-end of the receptor: 5′-CCCACGGCATCCCAGCGG-3′. The sense sequence was used as a control. The oligodeoxynucleotides were synthesized with a phosphorothioate backbone using an Applied Biosystems 394 DNA Sequencer and purified using HPLC (Molecular Biology Resource Facility, University of Oklahoma Health Sciences Center, Oklahoma City, Okla.).

In order to administer the oligodeoxynucleotides, Male, Swiss Webster mice were surgically implanted with chronic, indwelling guide cannula with their tips in the left lateral ventricle. To knock down sigma receptors with the McGill antisense, the mice were injected intracerebroventricularly every 12 hours, for a total of four times with 10 nmol/5 μl of the antisense oligodeoxynucleotide, its sense sequence, or an equivalent volume of saline (Kitaichi et al., 1997). Twelve hours after the last intracerebroventricular administration, the mice were challenged with a convulsive dose of cocaine (60 mg/kg, i.p.) and observed for the next 30 min for the onset of convulsions. Following behavioral testing, the intracerebroventricular injection sites were confirmed histologically and the brains were frozen for later confirmation of receptor knockdown. Fisher's exact test revealed a significant attenuation of cocaine-induced convulsions in mice who were pretreated with the McGill antisense (P<0.05).

The NYU and McGill antisense oligodeoxynucleotides were designed to interfere with the synthesis of sigma receptors, thus reducing the number of target sites through which cocaine can act. Both antisense sequences were designed to specifically target sigma receptors, although they act via different regions of the mRNA sequence. Thus, the ability of both antisense to attenuate the convulsive effects of cocaine provides strong confirmation that the anti-cocaine effects of the sigma ligands claimed herein produce their actions through antagonism of sigma receptors. Since the antisense oligodeoxynucleotides attenuate the convulsive effects of cocaine, they were also tested for their ability to attenuate the locomotor stimulatory effects of cocaine in mice. The effects of the antisense on locomotor activity were first tested alone, then in combination with a psychomotor stimulatory dose of cocaine.

FIG. 31 shows that treatment with either the NYU or McGill antisense oligodeoxynucleotides has no effects on basal locomotor activity. Male, Swiss Webster mice were treated intracerebroventricularly with the NYU or McGill antisense sequences to knock down σ₁ receptors as described for the convulsion studies. As controls, some mice received the corresponding sense sequences or saline vehicle. On the testing day, just prior to the cocaine challenge, the baseline locomotor activity of the animals was measured for 30 min using an automated activity monitor (San Diego Instruments, San Diego, Calif.). Horizontal locomotor activity was quantified for each animal as the number of disruptions in the 4×4 photobeam array that surrounded the plexiglas enclosures. ANOVA revealed no significant difference between the groups treated with saline, sense or antisense, indicating that alone, the treatments did not affect basal locomotor activity.

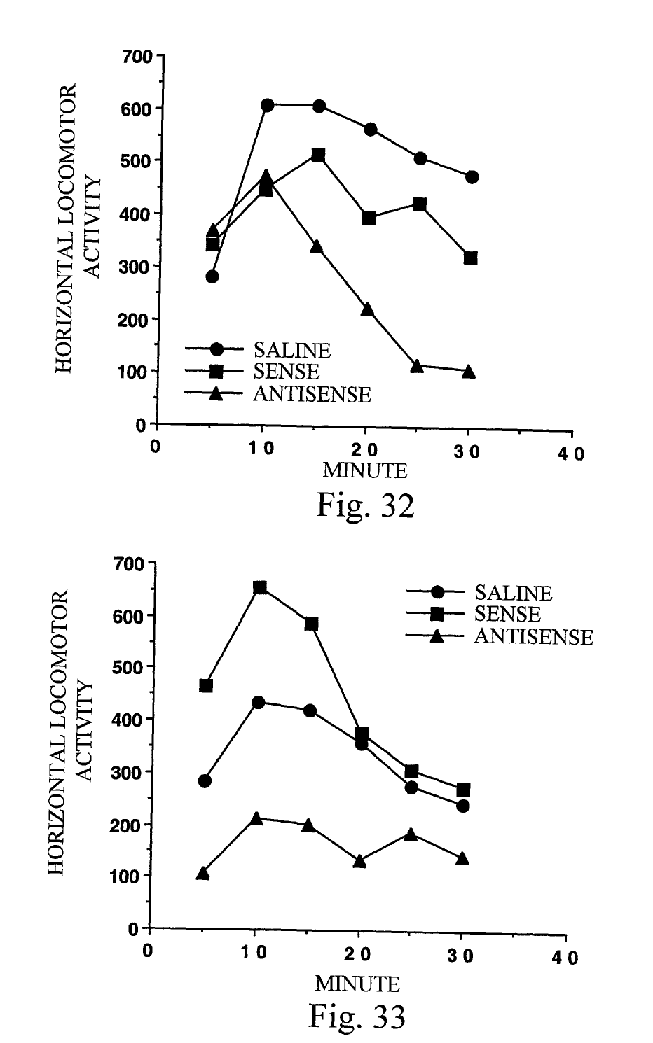

FIG. 32 shows that the NYU antisense against σ₁ receptors attenuates the locomotor stimulatory effects of cocaine. Male, Swiss Webster mice were injected intracerebroventricularly with the antisense oligodeoxynucleotide (10 μg/5 μl) on Days 1, 2 and 4 (King et al., 1997). As controls, the sense sequence or saline vehicle was administered using the same dosing schedule. On Day 5, the mice were acclimated for 30 min to the plexiglas enclosures of an automated activity monitor (San Diego Instruments, San Diego, Calif.). The mice were then injected with a locomotor stimulatory dose of cocaine (10 mg/kg, i.p.) and horizontal locomotor activity was quantified for the next 30 min by a computer as the number of disruptions in the 4×4 photobeam array the surrounded each enclosure. One hour after behavioral testing, the intracerebroventricular injection sites were confirmed histologically and the brains were frozen for later confirmation of receptor knockdown. Analysis of variance revealed a significant difference between the level of cocaine-induced locomotor activity among the treatment groups (P<0.05).

FIG. 33 shows that the McGill antisense oligodeoxynucleotide against σ₁ receptors attenuates the locomotor stimulatory effects of cocaine. Male, Swiss Webster mice were injected intracerebroventricularly every 12 hours, for a total of four times with 10 nmol/5 μl of the antisense oligodeoxynucleotide, its sense sequence, or an equivalent volume of saline (Kitaichi et al., 1997). Twelve hours after the last intracerebroventricular administration, the mice were acclimated for 30 min to the plexiglas enclosures of an automated activity monitor (San Diego Instruments, San Diego, Calif.). The mice were then injected with a locomotor stimulatory dose of cocaine (10 mg/kg, i.p.) and horizontal locomotor activity was quantified for the next 30 min by a computer as the number of disruptions in the 4×4 photobeam array the surrounded each enclosure. One hour after behavioral testing, the intracerebroventricular injection sites were confirmed histologically and the brains were frozen for later confirmation of receptor knockdown. Analysis of variance revealed a significant difference between the level of cocaine-induced locomotor activity among the treatment groups (P<0.05). Therefore, impeding cocaine's access to sigma receptors either by interfering with the synthesis of the target protein (antisense method) or by competing for access to existing receptors (pharmacological antagonism) attenuates the behavioral consequences and effects of cocaine.

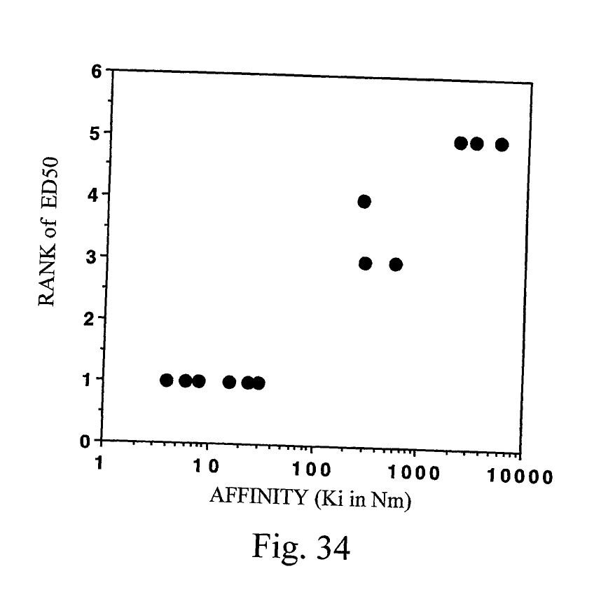

FIG. 34 shows the relationship between σ₁ binding and attenuation of cocaine-induced convulsions. In order to further evaluate the involvement of sigma receptors in the actions of cocaine, a series of aryl monosubstituted ligands that vary in their affinities for sigma receptors were tested. Male, Swiss Webster mice were injected (0-30 mg/kg, i.p.) with the following ligands that vary in their affinity for σ₁ receptors: high affinity nitro-substituted ligands (YZ027, YZ028 or YZ029), high affinity methoxy-substituted ligands (YZ011, YZ016, YZ018), moderate affinity amine-substituted ligands (YZ030, YZ032, YZ033), low affinity methoxy-amide-substituted ligands (YZ005, YZ007, YZ008). After a 15 min pretreatment period, the mice were challenged with a convulsive dose of cocaine (60 mg/kg, i.p.), and then observed for the next 30 min for the onset of convulsions. There was a relationship between the affinities of the compounds for σ₁ receptors and their ability to attenuate the convulsive effects of cocaine. High affinity ligands were most effective in preventing the cocaine-induced convulsions. Low affinity ligands were ineffective, and moderate affinity ligands provided intermediate protection. The ED₅₀ ranks correspond to the following ED₅₀ values: Rank 1<0.1 mg/kg; Rank 2 0.1-1 mg/kg; Rank 3 1-10 mg/kg; Rank 4 11-30 mg/kg; Rank 5>30 mg/kg.

### Materials and Methods

### Competition Binding Assays

Preparation of membranes. Crude P₂ membranes were prepared from guinea pig brain, rat brain, or rat liver using methods previously published (Bowen et al., 1993; Matsumoto et al., 1995, 1996). Briefly, the animals were decapitated and the brains and livers removed. The tissues were homogenized in ice-cold 10 mM Tris-sucrose buffer (0.32 M sucrose in 10 mM Tris-HCl, pH 7.4) in a volume of 10 ml/g wet tissue weight. The homogenates were centrifuged at 4° C. at 1000×g for 10 min and the supernatants were saved. The pellets were resuspended in 2 ml/g Tris-sucrose buffer and centrifuged at 4° C. at 1000×g for 10 min. The supernatants from both 1000×g spins were combined and centrifuged at 4° C. at 31,000×g for 15 min. The pellets were resuspended in 10 mM Tris-HCl, pH 7.4 in a volume of 3 ml/g and allowed to incubate for 30 min at 25° C. to lyse the membranes. Following the incubation, the suspensions were centrifuged at 4° C. at 31,000×g for 15 min. The pellets were resuspended in 10 mM Tris-HCl, pH 7.4 in a final volume of 1.53 ml/g tissue. Aliquots of tissue were stored at −80° C. until use. Protein content will be determined by the method of Bradford (Bradford, 1976) using a Bio-Rad protein assay kit and lyophilized bovine serum albumin standard (Hercules, Calif.).

### Sigma Receptor Assays

The methodological details were as previously published (Bowen et al., 1993; Matsumoto et al., 1995, 1996). Briefly, various concentrations of test ligand (0.05-100,000 nM) were incubated for 120 min at 25° C. in 50 mM Tris-HCl, pH 8.0 with 500 mg membrane protein, and 3 nM [³H](+)-pentazocine (for σ₁ assays) or 5 nM [³H]DTG plus saturating 1 mM dextrallorphan (for σ₁ assays); non-specific binding was determined in the presence of 10 mM haloperidol (Sigma, St. Louis, Mo.). The total reaction volume in each tube was 500 ml and the assays were run in duplicate or triplicate. The assays were terminated with 5 ml ice-cold 10 mM Tris-HCl, pH 8.0 and vacuum filtration using a Brandel cell harvester through glass fiber filters (Schleicher and Schuell, Keene, N.H.) pre-soaked for at least 30 min in 0.5% polyethyleneimine (Sigma, St. Louis, Mo.) to minimize non-specific binding to the filters. The filters were washed twice to further minimize non-specific binding.

### Non-sigma Assays

For all of the competition binding assays, the drugs were initially screened at 1000, 10,000 and 100,000 nM concentrations. If the compounds produced at least 30% displacement of the radioligand at the highest concentration, then full competition curves, consisting of at least 13 points, were constructed. If after three independent replications of the assay, the compounds did not display at least 30% inhibition at the 100,000 nM concentration, the affinities of the compounds were reported as >100,000 nM. The methodological details for the various radioligand binding assays followed protocols that have already been published or represent slight modifications of them and are outlined briefly below as well as the justification for testing these particular sites (de Costa et al., 1993; He et al., 1993; Matsumoto et al., 1995; Watson et al., 1986). Unless otherwise specified, the wash buffer were identical to the incubation buffer.

Historic “σ₁” ligands such as SKF 10,047 interact with opiate receptors, and a very early predecessor of the parent compound BD1008 was the kappa opiate receptor ligand U50,488. Therefore, the affinities of the novel ligands for opiate receptors were tested. Opiate receptors were labeled with 2 nM [³H](−)bremazocine (plus 100 nM DAMGO, 100 nM DSLET to block m and d receptors) in 10 mM Tris-HCl, pH 7.4; the membranes were incubated for 90 min at 25° C. and non-specific binding was determined in the presence 10 mM levallorphan.

The historic σ₁ ligand SKF 10,047 also interacts with PCP sites on the NMDA receptor. Therefore, to ensure that our novel s ligands lack this interaction, their affinities for these sites were measured. PCP sites were labeled with 5 nM [³H]TCP in 5 mM Tris-HCl, pH 7.4; the membranes were incubated for 60 min at 4° C. and non-specific binding was determined with 10 mM cyclazocine. Many σ ligands, including early generations of the compounds used herein have some affinity for muscarinic cholinergic receptors. Therefore, the affinities of our compounds for these sites were measured. Muscarinic M₁ receptors were labeled with 0.3 nM [³H]QNB in Krebs-Henseleit/HEPES buffer, pH 7.4; non-specific binding was determined in the presence of 10 mM QNB. After a 90 min incubation at 37° C., the membranes were washed with phosphate buffered saline, pH 7.4.

The historic “σ” ligand haloperidol is a well known antipsychotic drug. Since haloperidol and many other s-active antipsychotic drugs also interact with dopamine, adrenergic, and serotonergic receptors, the ability to the novel ligands to interact with these monoaminergic sites were tested. Dopamine D₂ receptors were labeled with 5 nM [³H](−)sulpiride in 50 mM Tris-HCl, pH 7.7 containing 120 mM NaCl; the membranes were incubated for 60 min at 25° C. and non-specific binding was determined with 1 mM haloperidol.

The affinities of the novel ligands for two subtypes of serotonergic receptors (5-HT₁ and 5-HT₂) were tested. 5-HT₁ receptors were labeled with 2 nM [³H]5-HT in 50 mM Tris-HCl, pH 7.7 containing 4 mM CaCl₂, 10 mM pargyline, and 0.1% ascorbic acid; non-specific binding was determined with 10 mM 5-HT. After a 30 min incubation at 25° C., the membranes were washed with 50 mM Tris-HCl, pH 7.4. 5-HT₂ receptors were labeled with 2 nM [³H]ketanserin in 50 mM Tris-HCl, pH 7.7; the membranes were incubated for 20 min at 37° C. and non-specific binding was determined with 1 mM methysergide.

The affinities of the novel ligands for three adrenergic receptor subtypes (a₁, a₂, b) were tested. α₁-Adrenoceptors were labeled with 3 nM [³H]prazosin in 50 mM Tris-HCl, pH 7.4; the membranes were incubated for 45 min at 30° C. and non-specific binding was determined with 10 mM phentolamine. α₂-Adrenoceptors were labeled with 2.5 nM [³H]clonidine in 50 mM Tris-HCl, pH 7.4; the membranes were incubated for 20 min at 25° C. and non-specific binding was determined in the presence of 10 mM yohimbine. β-Adrenoceptors were labeled with 1.5 nM [³H]dihydroalprenolol in 50 mM Tris-HCl, pH 7.8 containing 120 mM NaCl, 5 mM KCl, and 50 mM MgCl₂; the membranes were incubated for 30 min at 25° C. and non-specific binding was determined with 10 mM propranolol.

All of the assays were terminated with the addition of ice-cold buffer and vacuum filtration through glass fiber filters. Counts were extracted from the filters using Ecoscint cocktail (National Diagnostics, Manville, N.J., USA) for at least 8 hours prior to counting. IC₅₀ values were calculated using the GraphPad Prism program (San Diego, Calif.); goodness of fits for 1- vs. 2-sites were routinely evaluated. Ki values were calculated using the Cheng-Prusoff equation and Kd values that were previously determined in saturation experiments.

### Behavioral Effects of Cocaine

### Behavioral Toxic Effects

The dose response curves for cocaine-induced convulsions and lethality were determined by injecting male Swiss Webster mice with various doses of cocaine (0-150 mg/kg, i.p.). The animals were observed for the next 30 min for the occurrence of convulsions (operationally defined as clonic or tonic limb movements, which were sometimes accompanied by the loss of righting reflexes, wild running, and/or popcorn jumping; Matsumoto et al., 1997a; Ritz and George, 1997a,b; Witkin et al., 1993) or death. Due to the steepness of the dose response curves for the behavioral toxic effects of cocaine and the limited supply of some of the novel compounds, in the antagonism portions of the study, the ligands were tested for their ability to attenuate the behavioral toxic effects of single doses of cocaine that alone produced convulsions or lethality in 100% of animals.

Locomotor Effects

Male, Swiss Webster mice were acclimated for 30 min to the plexiglas enclosures of an automated activity monitoring system (San Diego Instruments, San Diego, Calif.). The mice were then injected with cocaine (0-20 mg/kg, i.p.) and horizontal locomotor activity was quantified by the computer for the next 30 min as the number of breaks made by the mice in the 4×4 photobeam array that surrounded each plexiglas enclosure. The dose of cocaine that produced the peak level of locomotor activity (10 mg/kg, i.p.) was selected as the challenge dose in subsequent antagonism testing with the sigma ligands.

### Effects of Sigma Ligands on Cocaine-Induced Convulsions: Systemic Administration

After an overdose of cocaine, many individuals convulse and it can be considered a sign of a potentially life threatening, but not necessarily fatal overdose. Male, Swiss Webster mice were pre-treated with one of the sigma ligands or controls (0-30 mg/kg, i.p.). After 15 min, the mice were administered a convulsive dose of cocaine (60 mg/kg, i.p.). The animals were then observed for the next 30 min for the occurrence of convulsions, which were operationally defined as clonic or tonic limb movements, which were sometimes accompanied by the loss of righting reflexes, wild running, and/or popcorn jumping.

To further probe the interaction of cocaine with putative agonists, mice were pre-treated with 30 mg/kg, i.p. of DTG, (+)-pentazocine, BD1031, or BD1052, followed 15 min later with a dose of cocaine (5-60 mg/kg, i.p.). The mice were then observed for the next 30 min for the onset of convulsions to determine whether the presence of the agonists produced a shift to the left of the cocaine dose curve, indicating increased behavioral toxicity.

### Effects of Sigma Ligands on Cocaine-Induced Lethality: Systemic Administration

### Pre-treatment Condition

Male, Swiss Webster mice were pre-treated with a sigma ligand or control (0-30 mg/kg, i.p.). After 15 min, the mice were administered a lethal dose of cocaine (125 mg/kg, i.p.). The mice were watched for the next 30 min and deaths were recorded. Those animals surviving the 30 min testing session were returned to their home cages where food and water were available, but they received no additional supportive therapies. Deaths after 24 hours were also noted to assess the longer term effects of the protection.

### Post-treatment Condition

Although the pre-treatments ensured that the σ receptors were occupied at the time of the cocaine overdose, in order to be of practical value, the antagonists must also be effective when administered after an overdose. Therefore, some of the antagonists that were effective under the pre-treatment condition were also administered after cocaine. The behavioral testing was conducted as described for the pre-treatments except that the mice were injected with the antagonists (or vehicle control) after the administration of a lethal dose of cocaine (125 mg/kg, i.p.) either: 1) immediately after the occurrence of the first convulsion, or 2) immediately before the onset of convulsions (i.e. the mice were running and falling over, but had not yet started seizing). The antagonists were administered relative to the onset of convulsions rather than at specified times after the administration of cocaine to facilitate the interpretation of the data. In terms of the behavioral sequelae that follow a lethal cocaine overdose, the onset of convulsions in our animals signifies that death may be imminent within a few minutes. Therefore, it is a significant physiological time point in the cascade that follows a lethal overdose. If the post-treatments were made at specified times after a cocaine overdose, it would be difficult to interpret the data at some of the intermediate time points due to variability in responsivity between animals. For example, 3 min after administration of cocaine, some animals would not yet have had a convulsion, others would have convulsed, and yet others would already be dead. Further, since in an emergency room, interventions are made as symptoms appear, the use of a clinically relevant time point (i.e. the onset of convulsions) at which to administer drugs is important. A dose of the antagonist that was effective under the pre-treatment condition was thus administered as a post-treatment. Similar to the pre-treatment studies, the animals were watched for 30 min for death, and survivors were checked again after 24 h.

### Effects of Sigma Ligands on Cocaine-Induced Locomotor Activity: Systemic Administration

### Effects of Sigma Ligands Alone

The effects of the sigma ligands were first evaluated to determine whether by themselves, they affected locomotor activity. Male, Swiss Webster mice were acclimated to the plexiglas enclosures of an automated activity monitoring system (San Diego Instruments, San Diego, Calif.). The mice were then injected with a sigma ligand or vehicle control (0-30 mg/kg, i.p.) and horizontal locomotor activity was quantified by the computer for the next 30 min as the number of breaks made by the mice in the 4×4 photobeam array that surrounded each plexiglas enclosure. Analysis of variance was used to determine whether the sigma ligands produced a level of locomotor activity that differed significantly from comparable injections of saline.

### Antagonism of Cocaine

For the antagonism experiments, the mice were acclimated to the activity monitors for 15 min. The animals were then injected with saline or a dose of sigma ligand that was determined to produce effects no different from saline when administered alone, except for BD1067 which had locomotor depressant actions on its own. After a 15 min pre-treatment period, a dose of cocaine that produced a peak level of locomotor activity (10 mg/kg, i.p.) was administered and horizontal locomotor activity was quantified for the subsequent 30 min. Analysis of variance was used to determine whether the sigma ligands significantly attenuated the locomotor stimulatory actions of cocaine.

### Effects of Antisense Treatment on Cocaine-Induced Convulsions and Locomotor Activity

Animals were surgically implanted with chronic indwelling guide cannula through which solutions could be administered intracerebroventricularly. For the surgeries, mice were deeply anesthetized with sodium pentobarbital (55 mg/kg, i.p.), preceeded by a preanesthetic dose of chlorpromazine (10 mg/kg, s.c.). Guide cannulae, constructed from 24 ga. stainless steel tubing, were implanted with their tips in the left lateral ventricle: 0.3 mm posterior, 0.7 mm lateral, and 2.4 mm ventral from bregma and the skull surface. Cannulae were secured to the skull surface with U-shaped wire and dental acrylic. Stainless steel stylets kept the cannulae sealed except during drug infusion.

The dosing schedule to knock down sigma receptors differed depending on the antisense that was administered and was based on those previously reported (King et al., 1997; Kitaichi et al., 1997). The dosing schedule for the NYU antisense was as previously reported by King et al. (1997). A total of three intracerebroventricular infusions (each 10 mg/5 ml) of the NYU antisense oligodeoxynucleotide were administered on Days 1, 2 and 4. As controls, the corresponding mismatch or sense sequence or saline was administered using the same regimen. On Day 5, the NYU-treated mice were evaluated behaviorally after being challenged with convulsive (60 mg/kg, i.p.) or locomotor stimulatory (10 mg/kg, i.p.) doses of cocaine as described above. The dosing schedule for the McGill antisense was as previously reported by Kitaichi et al. (1997). The mice were injected intracerebroventricularly every 12 hours, for a total of four times with 10 nmol/5 ml of the McGill antisense oligodeoxynucleotide, its sense sequence, or an equivalent volume of saline. Twelve hours after the last intracerebroventricular administration, the McGill-treated mice were challenged with convulsive (60 mg/kg, i.p.) or locomotor stimulatory (10 mg/kg, i.p.) doses of cocaine as described above.

Following the behavioral assessments, the cannula placements were histologically confirmed. The mice were sacrificed with an overdose of pentobarbital, cresyl violet dye (5 ml) was infused into the cannula, and the brains were removed. Coronal knife cuts were made through the site of penetration of the cannula and at the level of the cerebellum; the lateral and fourth ventricles were then examined for the presence of cresyl violet dye. Only those animals with injections histologically confirmed in the ventricles were used in the data analysis.

### Statistics

The data from the binding assays were analyzed using GraphPad Prism (San Diego, Calif., USA). The apparent Kᵢ values of the novel ligands were calculated using the Cheng-Prusoff equation and K_{d} values previously determined (Bowen et al., 1993; Hellewell et al., 1994; Matsumoto et al., 1990b).

The data from the behavioral studies were analyzed with Fisher's exact tests (GraphPad InStat, San Diego, Calif., USA). For all of the statistical analyses, P<0.05 was considered statistically significant. ED₅₀ values for the protective effects were calculated from the linear portion of the dose curves (GraphPad InStat, San Diego, Calif., USA).

Thus, it should be apparent that there has been provided in accordance with the present invention sigma receptor ligand compounds and methods for using these sigma receptor ligand compounds in the treatment of cocaine overdose and/or addiction that satisfy the objectives and advantages set forth above. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. A method of treating cocaine overdose and addiction which comprises administering to a patient an effective amount of a compound selected from the group represented by the formulas: 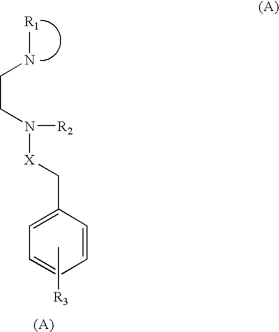
wherein R₁ is —(CH₂)ₙ— where n is an integer from 1 to 9, R₂ is methyl, H, ethyl, n-propyl, or allyl, X is
CH₂, and R₃ is 3,4 dichloro, m-methoxy, p-methoxy, o-methoxy, m-nitro, o-nitro, p-nitro, p-amine, o-amine, or m-amine, 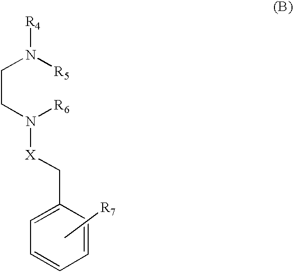
wherein R₄ is H, methyl, ethyl, n-propyl, or allyl, R₅ is H, methyl, ethyl, n-propyl, or allyl, R₆ is methyl, H, ethyl, n-propyl, or allyl, X is CH₂, and R₇ is 3,4 dichloro, m-methoxy, p-methoxy, o-methoxy, m-nitro, o-nitro, p-nitro, p-amine, o-amine, or m-amine, 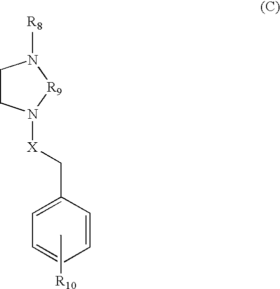
wherein R₈ is H, methyl, ethyl, n-propyl, or allyl, R₉ is —(CH₂)₂—, X is CH₂, and R₁₀ is 3,4-dichloro, m-methoxy, p-methoxy, o-methoxy, m-nitro, o-nitro, p-nitro, p-amine, o-amine, or m-amine, 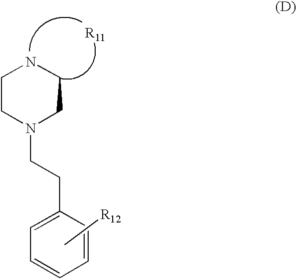
wherein R₁₁ is —(CH₂)ₙ— where n is an integer from 1 to 9, and R₁₂ is 3,4-dichloro, m-methoxy, p-methoxy, o-methoxy, m-nitro, o-nitro, p-nitro, p-amine, o-amine, or m-amine, 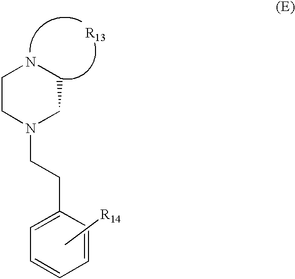
wherein R₁₃ is —(CH₂)ₙ— where n is an integer from 1 to 9, and R₁₄ is 3,4-dichloro, m-methoxy, p-methoxy, o-methoxy, m-nitro, o-nitro, p-nitro, p-amine, o-amine, or m-amine, 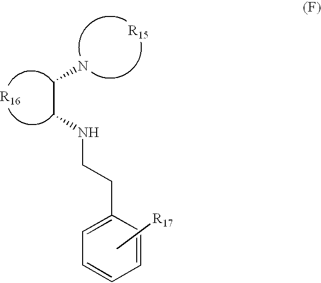
wherein R₁₅ is —(CH₂)ₙ— where n is an integer from 1 to 9, R₁₆ is —(CH₂)ₙ— where n is an integer from 1 to 9, and R₁₇ is 3,4-dichloro, m-methoxy, p-methoxy, o-methoxy, m-nitro, o-nitro, p-nitro, p-amine, o-amine, or m-amine, 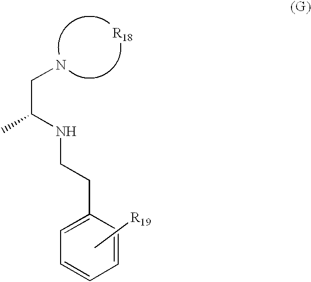
wherein R₁₈ is —(CH₂)ₙ— where n is an integer from 1 to 9, and R₁₉ is 3,4-dichloro, m-methoxy, p-methoxy, o-methoxy, m-nitro, o-nitro, p-nitro, p-amine, o-amine, or m-amine, 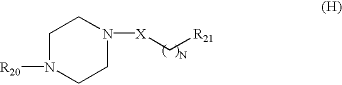
wherein R₂₀ is Ph(CH₂)₃ or m-methoxy-phenylethyl, n is an integer from 1 to 5, X is CH₂ or C═O, and R₂₁ is NH₂, 3,4-dichlorophenyl, m-methoxyphenyl, p-methoxyphenyl, o-methoxyphenyl, m-nitrophenyl, o-nitrophenyl, p-nitrophenyl, p-aminophenyl, o-aminophenyl, or m-aminophenyl,
as well as the isomers of these formulas and the pharmaceutically acceptable acid addition salts thereof.

2. A method of treating a human so as to stop the lethal effects of a cocaine overdose or treat the effects of cocaine addiction, wherein the method comprises administering to said human at least one compound selected from the group of compounds represented by the formulas: 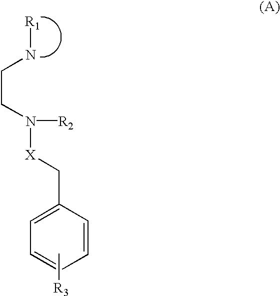
wherein R₁ is —(CH₂)ₙ— where n is an integer from 1 to 9, R₂ is methyl, H, ethyl, n-propyl, or allyl, X is
CH₂, and R₃ is 3,4 dichloro, m-methoxy, p-methoxy, o-methoxy, m-nitro, o-nitro, p-nitro, p-amine, o-amine, or m-amine, 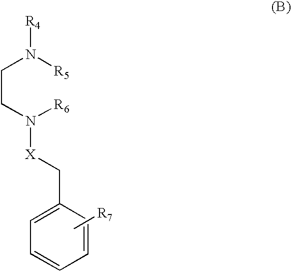
wherein R₄ is H, methyl, ethyl, n-propyl, or allyl, R₅ is H, methyl, ethyl, n-propyl, or allyl, R₆ is methyl, H, ethyl, n-propyl, or allyl, X is CH₂, and R₇ is 3,4 dichloro, m-methoxy, p-methoxy, o-methoxy, m-nitro, o-nitro, p-nitro, p-amine, o-amine, or m-amine, 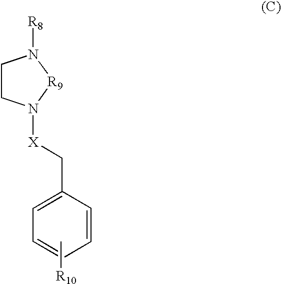
wherein R₈ is H, methyl, ethyl, n-propyl, or allyl, R₉ is —(CH₂)₂—, X is CH₂, and R₁₀ is 3,4-dichloro, m-methoxy, p-methoxy, o-methoxy, m-nitro, o-nitro, p-nitro, p-amine, o-amine, or m-amine, 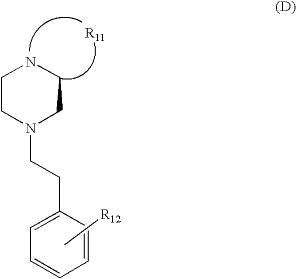
wherein R₁₁ is —(CH₂)ₙ— where n is an integer from 1 to 9, and R₁₂ is 3,4-dichloro, m-methoxy, p-methoxy, o-methoxy, m-nitro, o-nitro, p-nitro, p-amine, o-amine, or m-amine, 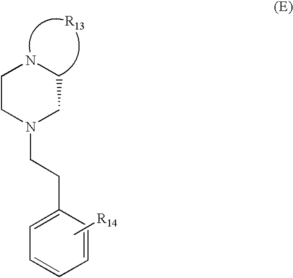
wherein R₁₃ is —(CH₂)ₙ— where n is an integer from 1 to 9, and R₁₄ is 3,4-dichloro, m-methoxy, p-methoxy, o-methoxy, m-nitro, o-nitro, p-nitro, p-amine, o-amine, or m-amine, 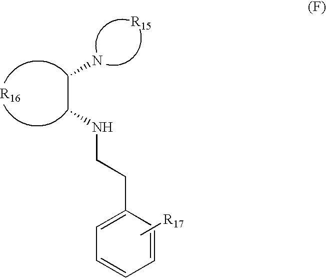
wherein R₁₅ is —(CH₂)ₙ— where n is an integer from 1 to 9, R₁₆ is —(CH₂)ₙ— where n is an integer from 1 to 9, and R₁₇ is 3,4-dichloro, m-methoxy, p-methoxy, o-methoxy, m-nitro, o-nitro, p-nitro, p-amine, o-amine, or m-amine, 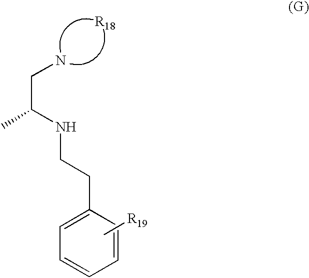
wherein R₁₈ is —(CH₂)ₙ— where n is an integer from 1 to 9, and R₁₉ is 3,4-dichloro, m-methoxy, p-methoxy, o-methoxy, m-nitro, o-nitro, p-nitro, p-amine, o-amine, or m-amine, 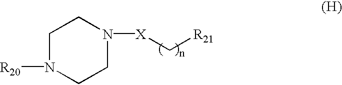
wherein R₂₀ is Ph(CH₂)₃ or m-methoxy-phenylethyl, n is an integer from 1 to 5, X is CH₂ or C═O, and R₂₁ is NH₂, 3,4-dichlorophenyl, m-methoxyphenyl, p-methoxyphenyl, o-methoxyphenyl, m-nitrophenyl, o-nitrophenyl, p-nitrophenyl, p-aminophenyl, o-aminophenyl, or m-aminophenyl,
as well as the isomers of these formulas and the pharmaceutically acceptable acid addition salts thereof; and
wherein the at least one compound is administered to said human in an amount sufficient to stop the lethal effects of a cocaine overdose or treat the effects of cocaine addiction in said human to thereby inhibit said effects.
